(19) United States
(12) Patent Application Publication
(10) Pub. No.: US 2026/0015376 A1
(43) Pub. Date: Jan. 15, 2026

(54) CONTINUOUS FLOW METHODS FOR PRODUCING MANNOSE-1-PHOSPHATE, POLYMORPHS OF MANNOSE-1-PHOSPHATE, AND COMPOSITIONS AND USES RELATED THERETO

(71) Applicant: Glycomine, Inc., San Carlos, CA (US)

(72) Inventors: Geoffrey S. HIRD, Chapel Hill, NC (US); Jeffrey P. WHITTEN, Santee, CA (US); Lili HAN, Shanghai (CN); Yongjie YU, Shanghai (CN); Ping HUANG, Shanghai (CN); Ruiheng ZHU, Shanghai (CN); Zhenbing ZHAO, Shanghai (CN); Hongwei YANG, Shanghai (CN); Pengjian LIU, Shanghai (CN); Xiaoyang WANG, Shanghai (CN)

(73) Assignee: Glycomine, Inc., San Carlos, CA (US)

(21) Appl. No.: 18/866,947

(22) PCT Filed: May 17, 2023
(86) PCT No.: PCT/US2023/022561

§ 371 Date: Nov. 18, 2024

Related U.S. Application Data
(60) Provisional application No. 63/343,447, filed on May 18, 2022.

(51) Int. Cl. C07H 11/04; A61K 9/1271; A61K 31/7024; C07H 1/02

(52) U.S. Cl. CPC C07H 11/04; A61K 9/1271; A61K 31/7024; C07H 1/02

(57) ABSTRACT

Provided herein are methods of synthesizing and compositions comprising an alpha isomer of mannose-1-phosphate (M1P). For example, such methods employ techniques in flow chemistry to achieve a substantially pure alpha isomer of M1P. Also provided are compositions, including pharmaceutical compositions, comprising substantially pure alpha isomer of M1P. Provided herein are also polymorphic forms of M1P, including polymorphic forms that may be produced from the substantially pure alpha isomer of M1P.

### CROSS-REFERENCE TO RELATED APPLICATION

This application is a U.S. national stage application under 35 U.S.C. § 371 of International Application No. PCT/US2023/022561, filed internationally on May 17, 2023, which claims the benefit of U.S. Provisional Application No. 63/343,447, filed May 18, 2022, which is hereby incorporated by reference in its entirety.

### FIELD

The present disclosure relates generally to mannose-1-phosphate (“M1P”), and more specifically to continuous flow methods to produce M1P, including particular polymorphs of M1P, and compositions comprising M1P produced using such continuous flow methods.

### BACKGROUND

Glycosylation, the enzymatic attachment of carbohydrates (glycans) to proteins and lipids, is a co-translational and post-translational modification (PTM) that is more common than any other PTM as it applies to a majority of proteins synthesized in the rough endoplasmic reticulum (ER). Glycosylation plays a critical role in a variety of biological processes of membrane and secreted proteins. In the ER, glycosylation defines the protein structure and folding and acts as a quality control mechanism that dictates the export of properly folded proteins to Golgi or targets misfolded ones for degradation. Glycan moieties may also act as ligands for cell surface receptors to mediate cell attachment or stimulate signal transduction pathways. Congenital disorders of glycosylation, also known as CDG syndromes, are a group of rare genetic diseases where tissue proteins and/or lipids carry defective glycosylation and/or lack of glycosylation. These diseases are linked to numerous enzymatic deficiencies and often times cause severe, sometimes fatal, impairments of the nervous system, muscles, intestines, and several other organ systems.

Common clinical symptoms in children with CDG include hypotonia, developmental delay, failure to thrive, hepatic dysfunction, coagulopathy, hypothyroidism, esotropia, abnormal fat pattern and inverted nipples, hypoglycemia, seizure, cerebellar hypoplasia, and stroke-like episodes in a developmentally delayed child. At an older age, in adolescence or adulthood, the presentation may include ataxia, cognitive impairment, the absence of puberty in females, small testes in males, retinitis pigmentosa, scoliosis, joint contractures, and peripheral neuropathy.

CDG may be classified into two groups: CDG type I and CDG type II. CDG type I is characterized by defects in the initial steps of N-linked protein glycosylation, i.e., biosynthesis of dolichol pyrophosphate linked oligosaccharide (DLO), which occur in the ER, or transfer of the DLO to asparagine residues of nascent polypeptides. CDG type II involves defects in further processing (synthetic or hydrolytic) of the protein-bound glycan. Currently, twenty-two CDG type I and fourteen type II variants have been identified. One of the most common subtypes of CDG is CDG-Ia (approximately 70% of all CDG cases), which is characterized by loss or reduction of phosphomannomutase 2 (PMM) activity leading to the deficiency or insufficiency in intracellular N-glycosylation (Jaeken et al. J. of Inherit. Met. Disease. 2008, 31: 669-672). PMM is responsible for the conversion of mannose-6-phosphate to mannose-1-phosphate (“M1P”).

Patients suffering from a reduction in PMM activity have reduced productions of M1P, associated with symptoms of multivisceral impairments. In order to overcome PMM production deficiency, it is important to supply downstream enzymes with the required substrate (i.e., M1P). However, the delivery and maintenance of such a systemic supply of M1P is problematic, as extracellular enzymes within bodily fluids degrade M1P when delivered exogenously by oral or intravenous administration. Another problem with exogenously delivered M1P is that its high polarity prevents M1P from penetrating into the cell interior (i.e., cytosol) and thus treating the deficiency in PMM production.

Potential solutions include the use a delivery vehicle, such as lipid particles, to encapsulate and deliver M1P to the cell interior (see WO 2015/053910 and WO 2020/205530). However, to make such formulations feasible on a commercial scale, commercially feasible methods to produce M1P on a large scale are needed. Current methods to produce M1P are generally limited due to the difficulty in obtaining the desired isomer of M1P in pure form and on a commercially viable and economical scale. During conventional production of M1P, synthetic intermediates degrade to beta anomeric forms, which results in a mixture of alpha and beta anomers in the final product composition. Isolating the pure, alpha isomer of M1P requires expensive and time-consuming purification methods.

Thus, what is needed in the art are commercially feasible methods to produce M1P on a large scale.

### BRIEF SUMMARY

In a first aspect, provided is a continuous flow production method, comprising:
a) providing a compound of Formula B-α-1 and a nucleophilic catalyst at a temperature between −30° C. and −10° C. to yield a precooled solution, wherein the compound of Formula B-α-1 is:

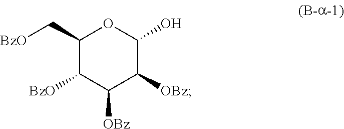

wherein the precooled solution has less than 1% of a compound of Formula (B-β-1):

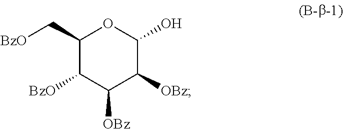

b) continuously combining the precooled solution and a phosphorylating agent in a first reactor at a temperature between −30° C. and 5° C. and a residence time between 30 seconds and 5 minutes, to produce an intermediate composition comprising a compound of Formula (C-α-1):

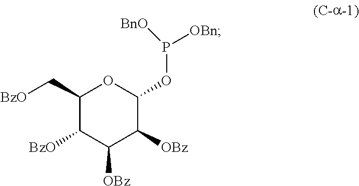

c) transferring the intermediate composition from the first reactor to a second reactor; and
d) continuously combining the intermediate composition with an oxidant in the second reactor at a temperature between 0° C. and 30° C. and for a residence time between 10 minutes and 30 minutes to produce a reaction mixture comprising a compound of Formula (D-α-1):

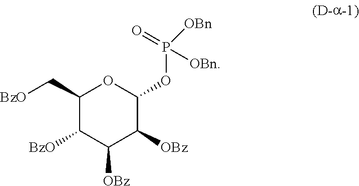

In some embodiments, the method further comprises deprotecting the compound of Formula (D-α-1) to produce a composition comprising an alpha isomer of mannose-1-phosphate, or a salt thereof, or a hydrate of any of the foregoing, wherein less than 2% of the composition is the beta isomer of mannose-1-phosphate, or a salt thereof, or a hydrate of any of the foregoing.

In another aspect, provided is a composition produced according to the method described above. In some embodiments, provided is a composition comprising an alpha isomer of mannose-1-phosphate, or a salt thereof, or a hydrate of any of the foregoing, wherein the purity of the composition is at least 96%. In some embodiments, less than 2% of the composition is the beta isomer of mannose-1-phosphate, or a salt thereof, or a hydrate of any of the foregoing.

In another aspect, provided is a method of producing a crystalline Form C of mannose-1-phosphate, comprising: combining a starting composition comprising (i) substantially pure alpha isomer of mannose-1-phosphate, or a salt thereof, or a hydrate of any of the foregoing, or (ii) crystalline Form A of a substantially pure alpha isomer of mannose-1-phosphate, or a salt thereof, or a hydrate of any of the foregoing, or (iii) crystalline Form B of a substantially pure alpha isomer of mannose-1-phosphate, or a salt thereof, or a hydrate of any of the foregoing, or any combination of (i)-(iii), with a solvent comprising water and alcohol to produce a suspension; and stirring the suspension to produce a product composition comprising crystalline Form C of mannose-1-phosphate.

In another aspect, provided is a crystalline Form C of mannose-1-phosphate produced according to the method described in the previous paragraph. In some embodiments, provided is a crystalline Form C of mannose-1-phosphate that is a substantially pure alpha isomer of a potassium trihydrate salt of mannose-1-phosphate.

In another aspect, provided is a composition comprising at least 98% by weight of a crystalline Form C according to the description above.

In another aspect, provided is a composition, comprising: (i) a liposome comprising one or more phospholipids conjugated to polyethylene glycol (PEG); and (ii) alpha isomer of mannose-1-phosphate, or a salt thereof, or a hydrate of any of the foregoing, encapsulated in the liposome.

In one embodiment, provided is a composition, comprising: liposomes having a lipid membrane enclosing an intraliposomal compartment, wherein the liposomes encapsulate in the intraliposomal compartment an alpha isomer of mannose-1-phosphate, or a salt thereof, or a hydrate of any of the foregoing, and wherein the lipid membrane comprises: (a) at least one phospholipid having an ethanolamine head group and at least one unsaturated fatty acid tail; (b) at least one phospholipid having a choline group and at least one unsaturated fatty acid tail; and (c) at least one phospholipid having an ethanolamine head group and at least one saturated fatty acid tail, conjugated to polyethylene glycol (PEG); and intraliposomal buffer comprising a buffer salt and optionally acid, wherein the pKa of the buffer salt is between 6 to 8.5; extraliposomal buffer comprising a buffer salt and a tonicity modifier, wherein the pKa of the buffer salt is between 6 to 8.5; and optionally a radical scavenging antioxidant.

In yet other aspects, provided is a method for treating a congenital disorder of glycosylation (CDG) in a subject in need thereof, by administering any of the M1P compositions (including pharmaceutical compositions) provided herein to the subject.

### DESCRIPTION OF THE FIGURES

The present application can be understood by reference to the following description taken in conjunction with the accompanying figures.

FIG. 1 depicts an exemplary scheme to produce a M1P salt, or a hydrate thereof, from (2S,3S,4S,5S,6R)-6-(hydroxymethyl)tetrahydro-2H-pyran-2,3,4,5-tetraol.

FIG. 2 depicts an exemplary method and system to manufacture (2R,3R,4S,5S,6R)-2-((benzoyloxy)methyl)-6-((bis(benzyloxy)phosphoryl)oxy)tetrahydro-2H-pyran-3,4,5-triyl tribenzoate, which can be deprotected to produce M1P, or a salt or hydrate thereof.

FIG. 3 depicts an exemplary XRPD of crystalline Form A, a dihydrate, dipotassium salt of M1P.

FIG. 4 depicts an exemplary XRPD of crystalline Form B, a trihydrate, dipotassium salt of M1P.

FIG. 5 depicts an exemplary XRPD of crystalline Form C, a trihydrate, dipotassium salt of M1P.

FIG. 6 depicts an exemplary TGA spectrum of crystalline Form C.

### DETAILED DESCRIPTION

The following description sets forth exemplary compositions, methods, parameters and the like. It should be recognized, however, that such description is not intended as a limitation on the scope of the present disclosure but is instead provided as a description of exemplary embodiments.

In some aspects, provided herein are methods that involve continuous flow chemistry to produce pure or substantially pure alpha isomer of mannose-1-phosphate (including salts and hydrates thereof). In some variations, the composition produced has less than 2% of the beta isomer of mannose-1-phosphate (including salts and hydrates thereof). In other aspects, provided herein are compositions comprising pure or substantially pure alpha isomer of mannose-1-phosphate (including salts and hydrates thereof), as well as liposomal formulations incorporating such pure or substantially pure alpha isomer of mannose-1-phosphate (including salts and hydrates thereof), and uses of such formulations. In some embodiments, the purity of a target compound is the amount of that compound (including salts and hydrates thereof) as compared to all other components in the sample, expressed as a percentage. In some embodiments, sample purity is measured by HPLC, including for example, using HPLC separation methods to separates each component into separate peaks on a chromatogram. The peak of area of the target compound is compared to the peak area of all other components, and purity is expressed as a percentage value.

### Continuous Flow Production Method

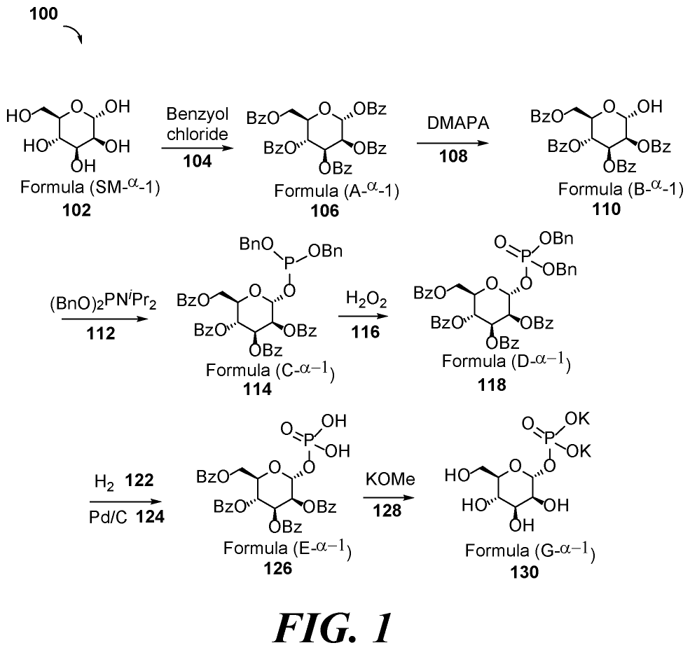

With reference to FIG. 1, process 100 is an exemplary scheme to produce Form C of M1P. Form C can be produced starting from (2S,3S,4S,5S,6R)-6-(hydroxymethyl)tetrahydro-2H-pyran-2,3,4,5-tetraol (compound 102). In the first step of process 100, the alcohol groups of compound 102 are protected using a suitable protecting agent, such as benzoyl chloride (reagent 104), to produce (2R,3S,4S,5R,6R)-6-((benzoyloxy)methyl)tetrahydro-2H-pyran-2,3,4,5-tetrayl tetrabenzoate (compound 106) under suitable reaction conditions. For example, the reaction may be performed at a temperature between about 10° C. and 30° C., and in the presence of a suitable base such as pyridine. Suitable reaction times may be for about three hours. In other embodiments, other suitable protecting agents, solvents, and bases may be used. For example, the reaction may be performed in acetonitrile, using a sufficient quantity of a base, such as diisopropylethylamine.

With reference again to process 100 (FIG. 1), compound 106 may be reacted with a suitable base, such as DMAPA (reagent 108), in a suitable solvent, such as THF, to selectively remove the benzoyl group of the oxygen attached to the anomeric center, to yield (2R,3R,4S,5S,6S)-2-((benzoyloxy)methyl)-6-hydroxytetrahydro-2H-pyran-3,4,5-triyl tribenzoate (compound 110) under suitable reaction conditions. For example, the reaction may be performed at a temperature between about 60° C. and 65° C., for a sufficient quantity of time, such as 12 to 14 hours. In other embodiments, other bases and solvents may be used to effect selective deprotection.

With reference again to process 100 (FIG. 1), compound 110 may be phosphorylated under suitable flow chemistry conditions to produce (2R,3R,4S,5S,6R)-2-((benzoyloxy)methyl)-6-((bis(benzyloxy)phosphaneyl)oxy)tetrahydro-2H-pyran-3,4,5-triyl tribenzoate (compound 114). For example, compound 110 and a nucleophilic catalyst, such as DCI, may be dissolved in a suitable solvent, such as acetonitrile, at a temperature between about −30° C. and −10° C. to yield a first precooled solution. This precooled solution may then be continuously combined, at a temperature between about 0° C. and 5° C., with a second solution containing a suitable phosphorylating agent, such as (BnO)₂PNⁱPr₂ (reagent 112), for a sufficient time, to yield an intermediate composition comprising compound 114. The two solutions may be continuously combined for a total time between about 30 seconds and 5 minutes.

With reference again to process 100 (FIG. 1), the intermediate composition comprising compound 114 may be oxidized under suitable flow chemistry conditions to produce (2R,3R,4S,5S,6R)-2-((benzoyloxy)methyl)-6-((bis(benzyloxy)phosphoryl)oxy)tetrahydro-2H-pyran-3,4,5-triyl tribenzoate (compound 118). For example, the intermediate composition comprising compound 114 may be continuously combined, at a temperature between about 0° C. and 30° C., with a suitable oxidant, such as H₂O₂, to yield compound 118. The intermediate composition comprising compound 114 may be continuously combined with the oxidant for a total time between about 10 minutes and 30 minutes.

With reference again to process 100 (FIG. 1), compound 118 may be subjected to catalytic hydrogenation to selectively remove the benzyl protecting groups and produce (2R,3R,4S,5S,6R)-2-((benzoyloxy)methyl)-6-(phosphonooxy)tetrahydro-2H-pyran-3,4,5-triyl tribenzoate (compound 126). For example, compound 118 may be dissolved in a suitable solvent or combination of solvents, such as EtOAc and MeOH, and combined with Pd/C (reagent 124). This solution may then be reacted with gaseous H₂ (reagent 122) at a temperature between about 20° C. and 30° C., for a sufficient time, such as 10 to 12 hours, to yield compound 126.

With reference again to process 100 (FIG. 1), compound 126 may be deprotected under suitable conditions to produce (2R,3S,4S,5S,6R)-3,4,5-trihydroxy-6-(hydroxymethyl)tetrahydro-2H-pyran-2-yl dihydrogen phosphate (compound 130), or a salt or hydrate thereof. For example, compound 126 may be dissolved in a suitable solvent, such as MeOH, and reacted with a suitable base, such as KOMe (reagent 128), at a temperature between about 20° C. and 25° C., for a sufficient time, such as 20 to 22 hours, to yield compound 130, or a hydrate thereof.

In process 100, KOMe was used as the base in converting compound 126 to compound 130, which is a potassium salt. In other variations, other suitable salts may be used. It should be understood that the base used in this step determines the specific salt resulting from compound 126. For example, is NaOMe was used as the base, compound 126 would convert to the corresponding sodium salt.

It is desired to produce M1P or a salt or hydrate thereof as a substantially pure alpha isomer. Cyclic carbohydrates exist in either “alpha” or “beta” stereochemical forms, or isomers, depending on the position of the substituent attached to the anomeric center. Because they are isomers at the anomeric center, such forms are sometimes termed “anomers”. In an alpha anomer, the substituent attached to the anomeric carbon is on the opposite side of (i.e., trans to) the substituent attached to the other carbon adjacent to the ring oxygen. In beta anomers, the situation is reversed, and the substituent attached to the anomeric carbon is on the same side of (i.e., cis to) the substituent attached to the other carbon adjacent to the ring oxygen. The alpha and beta forms of M1P, in “chair” and “flat” views, are given below:

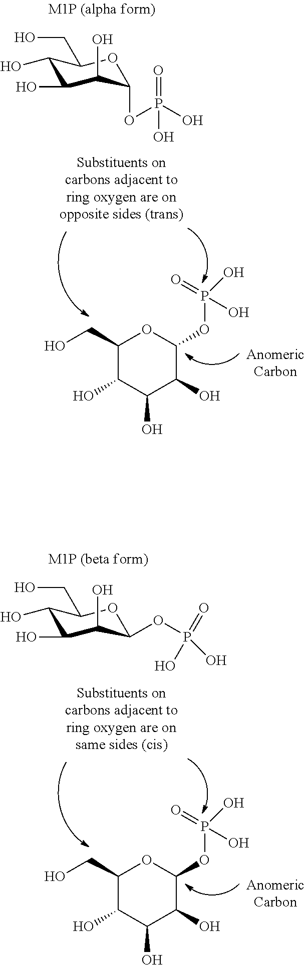

In some variations, a composition comprising substantially pure alpha isomer refers to a composition that has less than 5%, less than 4%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.7%, less than 0.6%, less than 0.5%, less than 0.4% less than 0.3%, less than 0.2%, or less than 0.1% of beta isomer of M1P. In one variation, a composition comprising substantially pure alpha isomer refers to a composition that does not have a detectable amount of beta isomer of M1P.

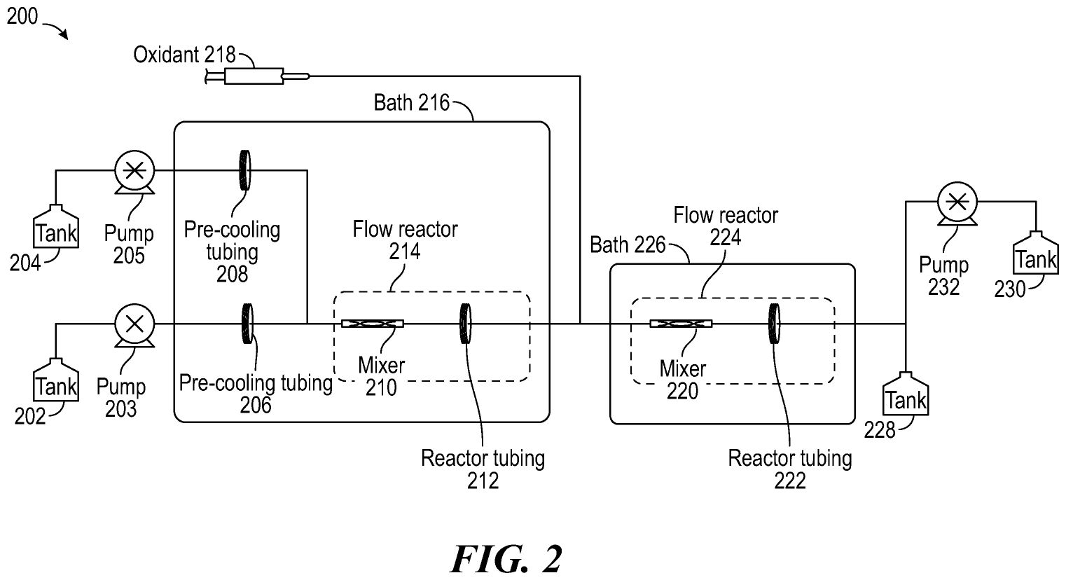

In order to produce substantially pure alpha isomer of M1P, in some embodiments, flow chemistry is applied to convert compound 110 to compound 118, and compound 118 can then be deprotected to produce a M1P salt or hydrate as described above. With reference to FIG. 2, depicted is exemplary system 200 to produce compound 118 from compound 110 in two steps using flow chemistry.

In the first flow chemistry step, compound 110 is converted to compound 114. Tank 202 contains compound 110 and a nucleophilic catalyst, such as DCI, in a suitable solvent, such as acetonitrile, at a temperature between about −30° C. and −10° C. Tank 204 contains reagent 112 in the same solvent. Tanks 202 and 204 each continuously feed, via pumps 203 and 205, into pre-cooling tubing 206 and 208, respectively, which are then combined in mixer 210 and passed through reactor tubing 212 at a temperature between about 0° C. and 5° C., which is maintained by bath 216. The total residence time in mixer 210 and reactor tubing 212, which together comprise flow reactor 214, is between about 30 seconds and 5 minutes. A composition comprising compound 114 is the product of this step, and is carried forward in the second flow chemistry step without isolation or purification.

In the second flow chemistry step, compound 114 is converted to compound 118. The composition comprising compound 114, and a suitable oxidant 218, are continuously combined in mixer 220 and passed through reactor tubing 222 at a temperature between about 0° C. and 30° C., which is maintained by bath 226. For example, the oxidant may be H₂O₂ (reagent 116 in FIG. 1). The total residence time in mixer 220 and reactor tubing 222, which together comprise flow reactor 224, is between about 10 minutes and 30 minutes. A reaction mixture comprising compound 118 is the product of this step, and is collected in tank 228.

Following the second flow chemistry step, the reaction mixture comprising compound 118 is quenched with an aqueous solution comprising a reducing agent. Tank 230 contains an aqueous solution of Na₂SO₃, or another suitable reducing agent, which is transferred to tank 228, which contains the reaction mixture comprising compound 118, via pump 232. The quenching is conducted at a temperature between about 0° C. and 25° C., for between about 1 minute and 15 minutes. Following the quench, the reaction mixture is diluted with a suitable organic solvent and compound 118 is isolated via extraction. In some embodiments, the organic solvent used in extraction is toluene.

Thus, in some aspects, provided is a continuous flow production method that comprises: a) providing a compound of Formula (B-α-1) and a nucleophilic catalyst at a temperature between −30° C. and −14° C. to yield a precooled solution; b) continuously combining the precooled solution and a phosphorylating agent in a first reactor at a temperature between 0° C. and 5° C. and a residence time between 30 seconds and two minutes, to produce an intermediate composition comprising a compound of Formula (C-α-1); c) transferring the intermediate composition from the first reactor to a second reactor; and d) continuously combining the intermediate composition with an oxidant in the second reactor at a temperature between 20° C. and 25° C. and for a residence time between 10 minutes and 20 minutes to produce a reaction mixture comprising a compound of Formula (D-α-1).

In some variations, the compound of Formula (B-α-1) is:

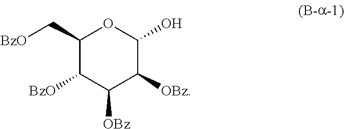

In some variations, the compound of Formula (C-α-1) is:

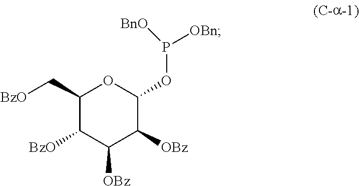

In some variations, the compound of (D-α-1) is:

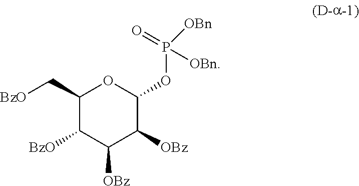

In some embodiments, in step a) of the continuous production method described above, suitable nucleophilic catalysts may include, for example, 1H-tetrazole, 5-(p-nitrophenyl)-1H-tetrazole, 5-ethylthio-1H-tetrazole, benzimidazolium triflate, a mixture of dimethylaminopyridine and 5-(p-nitrophenyl)-1H-tetrazole, a mixture of N-methylimidazole and 1H-tetrazole. In one variation, the nucleophilic catalyst is 4,5-dicyanoimidazole.

In some variations, in the precooled solution of step a), an organic solvent is used. In one embodiment, the compound of Formula (B-α-1) and the nucleophilic catalyst are provided in a suitable solvent, such as an organic solvent. In some embodiments, the organic solvent is a polar aprotic solvent. In some embodiments, the polar aprotic solvent is acetone, dichloromethane, dimethylformamide, dimethylpropyleneurea, dimethylsulfoxide, ethyl acetate, hexamethylphosphoric triamide, 2-MeTHF, or tetrahydrofuran. In one embodiment, wherein the organic solvent is acetonitrile, THF, or 2-MeTHF. In one variation, the solution comprises acetonitrile.

In some embodiments, in step a) of the continuous production method, the precooled solution comprising the compound of Formula (B-α-1) and the nucleophilic catalyst is held at a temperature between −30° C. and −10° C., between −30° C. and −15° C., between −30° C. and −20° C., between −20° C. and −10° C., between −20° C. and −15° C., or between −30° C. and −25° C.

In some embodiments, in step b) of the continuous production method described above, suitable phosphorylating agents may include, for example, 2-chloro-4,4,5,5-tetramethyl-1,3,2-dioxaphospholane, diethylphosphoramidous dichloride, tris(1-pyrrolidinyl)phosphine, 2-chloro-1,3,2-benzodioxaphosphorin-4-one, dibenzyl N,N-diethylphosphoramidite, diallyl N,N-diisopropylphosphoramidite, bis(2-cyanoethyl)-N,N-diisopropylphosphoramidite, 2-Cyanoethyl N,N,N′,N′-tetraisopropylphosphorodiamidite, or 2-cyanoethyl N,N-diisopropylchlorophosphoramidite. In one variation, the phosphorylating agent is dibenzyl N,N-diisopropylphosphoramidite, also referred to as (BnO)₂PNⁱPr₂. In some embodiments, the phosphorylating agent is at least partially dissolved in an organic solvent. In some embodiments, the organic solvent comprises polar aprotic solvent. In other embodiments, the organic solvent is acetonitrile, THF, or 2-MeTHF. In one variation, the organic solvent is acetonitrile.

In some embodiments, in step b) of the continuous production method, the precooled solution and the phosphorylating agent are continuously combined in the reactor at a temperature between −30° C. and 5° C., between −30° C. and 0° C., between −30° C. and −5° C., between −20° C. and 0° C., between −20° C. and −5° C., or between −20° C. and −10° C.

In some embodiments, in step b) of the continuous production method, the precooled solution and the phosphorylating agent are continuously combined in the reactor for a residence time between 30 seconds and 5 minutes, 30 seconds and 4 minutes, 30 seconds and 3 minutes, 30 seconds and 2 minutes, or 30 seconds and 1 minute; or for less than 5 minutes, less than 4 minutes, less than 3 minutes, less than 2 minutes or less than 1 minute.

In some embodiments, in step b) of the continuous production method, the precooled solution and the phosphorylating agent are each provided at a flow rate between 0.5 mL/min and 1 L/min. In some embodiments, in step b) of the continuous production method, the precooled solution is provided at a flow rate between 0.5 mL/min and 1 L/min. In some embodiments, the phosphorylating agent is transferred at a flow rate between 0.5 mL/min and 1 L/min.

The intermediate composition produced in step b) of the continuous production method comprises a compound of Formula (C-α-1). In certain embodiments, the intermediate composition comprising a compound of Formula (C-α-1) contains less than 1%, less than 0.5%, less than 0.1% or less than 0.01% of a compound of Formula (B-α-1). In some variations, the compound of Formula (B-α-1) is not detectable in the intermediate composition. In certain embodiments, the intermediate composition comprising a compound of Formula (C-α-1) contains less than 1%, less than 0.5%, less than 0.1% or less than 0.01% of a compound of Formula (B-3-1):

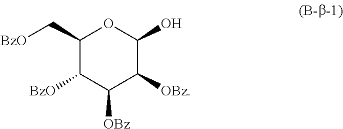

In some variations, the compound of Formula (B-α-1) is not detectable in the intermediate composition. In one variation, intermediate composition comprising a compound of Formula (C-α-1) contains less than 1% of a compound of Formula (B-α-1) and less than 1% of a compound of Formula (B-β-1).

In some embodiments, in step d) of the continuous production method described above, suitable oxidants include organic peroxides, for example, hydroperoxides, peroxy acids, peroxy esters, diacyl peroxides, and dialkylperoxides. In some embodiments, tert-butyl hydroperoxide, cumene hydroperoxide, or dicumyl peroxide may be used. In one variation, H₂O₂ is used. In some embodiments, between 1 and 3 molar equivalents of H₂O₂ are used. In some embodiments, the oxidant may be dissolved in a suitable organic solvent. In other embodiments, the oxidant is in an aqueous solution.

In some embodiments, in step d) of the continuous production method, the oxidant and the intermediate composition comprising a compound of Formula (C-α-1) are continuously combined in the reactor at a temperature between 0° C. and 30° C., between 5° C. and 30° C., between 10° C. and 30° C., between 15° C. and 30° C., between 20° C. and 30° C., or between 25° C. and 30° C.

In some embodiments, in step d) of the continuous production method, the oxidant and the intermediate composition comprising a compound of Formula (C-α-1) are continuously combined in the reactor for a residence time between 10 minutes and 30 minutes, 15 minutes and 30 minutes, 20 minutes and 30 minutes, 25 minutes and 30 minutes, or 10 minutes and 15 minutes; or for less than 30 minutes, less than 25 minutes, less than 20 minutes, less than 15 minutes or less than 12 minutes.

In some embodiments, in step d) of the continuous production method, the oxidant is provided at a flow rate between 0.5 mL/min and 1 L/min.

In some embodiments, the reaction mixture comprising a compound of Formula (D-α-1) contains less than 2%, less than 1%, less than 0.5%, less than 0.1% or less than 0.01% of a compound of Formula (D-β-1):

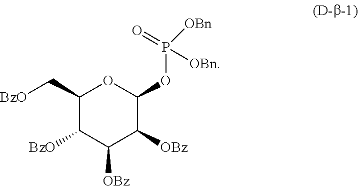

In one variation, the reaction mixture comprising a compound of Formula (D-α-1) contains less than 2% of a compound of Formula (D-β-1). In some variations, the compound of Formula (D-β-1) is not detectable in the reaction mixture.

In some embodiments, the reaction mixture comprising a compound of Formula (D-α-1) is quenched. In some embodiments, the reaction mixture is quenched with a solution comprising a reducing agent. In some variations, the solution is an aqueous solution. In some embodiments, the reducing agent is a dithionate. In other embodiments, the reducing agent is a thiosulfate. In one embodiments, the reducing agent is Na₂S₂O₃. In one variation, the reducing agent is Na₂SO₃. In some embodiments, the reducing agent is a metal catalyst. In one variation, the reducing agent is manganese dioxide. In another variation, the reducing agent comprises an Fe²⁺ ion. In some embodiments, the reducing agent is an iodide. In one variation, the reducing agent is potassium iodide. In another variation, the reducing agent is activated carbon. In some embodiments, the reducing agent is ascorbic acid. In some embodiments, the reaction mixture is quenched at a temperature between 0° C. and 25° C., between 0° C. and 20° C., between 0° C. and 15° C., between 0° C. and 10° C., between 0° C. and 5° C., or between 10° C. and 200° C. In one variation, the reaction mixture is quenched at a temperature between 0° C. and 25° C.

In another aspect of the invention, the compound of Formula (D-α-1) is extracted into an organic solvent. In some embodiments, the organic solvent comprises toluene. In some embodiments, the organic solvent is washed with water followed by a 10% solution of Na₂SO₄. In some embodiments, the compound of Formula (D-α-1) is isolated by evaporating at least a portion of the organic solvent. In some embodiments, the compound of Formula (D-α-1) is not subjected to further purification.

In one aspect of the invention, the compound of Formula (D-α-1) is deprotected to produce a composition comprising an alpha isomer of mannose-1-phosphate, or a salt thereof, or a hydrate of any of the foregoing, wherein less than 2% of the composition is the beta isomer of mannose-1-phosphate, or a salt thereof, or a hydrate of any of the foregoing. In some embodiments, less than 5%, less than 4%, less than 3%, or less than 1% of the composition is the beta isomer of mannose-1-phosphate, or a salt thereof, or a hydrate of any of the foregoing.

With reference again to FIG. 1, in some embodiments, the compound of Formula (B-α-1) is produced by reacting a compound of Formula (A-α-1) with DMAPA to selectively remove the benzoyl (Bz) group of the oxygen attached to the anomeric center. In some variations, the compound of Formula (A-α-1) is:

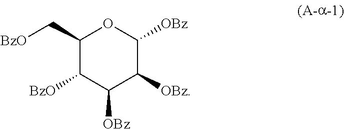

In some embodiments, the compound of Formula (B-α-1) is produced by reacting a compound of Formula (A-α-1) with an amine base to selectively remove the benzoyl (Bz) group of the oxygen attached to the anomeric center. In some embodiments, the amine base is DMAPA, N,N′-dimethyl-1,3-propanediamine, N,N-dimethyldipropylenetriamine, N¹-isopropyldiethylenetriamine, 3-(methylamino)propylamine, N,N,N′,N′-tetraethyldiethylenetriamine, 3,3′-iminobis(N,N-dimethylpropylamine), or diethylenetriamine. In one variation, the amine base is DMAPA.

In some embodiments, the compound of Formula (B-α-1) is produced by reacting a compound of Formula (A-α-1) with an amine base at a temperature between 40° C. and 70° C., between 50° C. and 75° C., between 60° C. and 75° C., or between 60° C. and 65° C. In one variation, the reaction is performed between 60° C. and 65° C.

In some embodiments, the compound of Formula (B-α-1) is produced by reacting a compound of Formula (A-α-1) with an amine base for a reaction time between 6 hours and 15 hours, 8 hours and 15 hours, 9 hour and 15 hours, 10 hours and 14 hours, 11 hours and 14 hours, or 12 hours and 14 hours; or for less than 17 hours, less than 16 hours, less than 15 hours, less than 14 hours or less than 13 hours. In one variation, the reaction time is between 12 and 14 hours.

With reference again to FIG. 1, in some embodiments, the compound of Formula (A-α-1) is produced by reacting a compound of Formula (SM-α-1) with benzoyl chloride to attach benzoyl groups to each hydroxyl group. In some variations, the compound of Formula (SM-α-1) is:

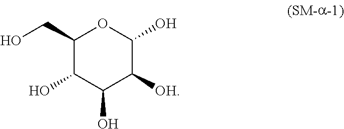 [SMILES: OC[C@H]1O[C@H](O)[C@@H](O)[C@@H](O)[C@@H]1O]

In some embodiments, the compound of Formula (A-α-1) is produced by reacting a compound of Formula (SM-α-1) with benzoyl chloride in the presence of a suitable base. In some embodiments, the base is an amine base. In some variations, the base serves as the reaction solvent. In some embodiments, the base is pyridine, piperidine, trimethylamine, diisopropylethylamine, ethylamine, ammonia, or aniline. In one variation, the base is pyridine.

In some embodiments, the compound of Formula (A-α-1) is produced by reacting a compound of Formula (SM-α-1) with benzoyl chloride at a temperature between 10° C. and 30° C., between 10° C. and 25° C., between 15° C. and 25° C., or between 20° C. and 25° C. In one variation, the reaction is performed between 20° C. and 25° C.

In some embodiments, the compound of Formula (A-α-1) is produced by reacting a compound of Formula (SM-α-1) with benzoyl chloride for a reaction time between 30 minutes and 3 hours, 45 minutes and 3 hours, 1 hour and 3 hours, 1.5 hours and 3 hours, 2 hours and 3 hours, 2.5 hours and 3 hours, or 3 hours and 5 hours; or for less than 10 hours, less than 8 hours, less than 6 hours, less than 5 hours or less than 4 hours. In one variation, the reaction time is 3 hours.

With reference again to FIG. 1, in some embodiments, the compound of Formula (E-α-1) is produced by reacting a compound of Formula (D-α-1) with hydrogen in the presence of Pd/C to selectively deprotect the phosphate group. In some variations, the compound of Formula (E-α-1) is:

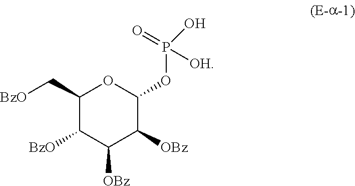

In some embodiments, the compound of Formula (E-α-1) is produced by reacting a compound of Formula (D-α-1) with hydrogen and Pd/C, wherein the Pd/C used is 5% w/w, 10% w/w, 15% w/w, or 20% w/w. In one variation, the Pd/C used is 15% w/w.

In some embodiments, the compound of Formula (E-α-1) is produced by reacting a compound of Formula (D-α-1) with hydrogen and Pd/C at a temperature between 15° C. and 30° C., between 20° C. and 30° C., or between 25° C. and 30° C. In one variation, the reaction is performed between 20° C. and 30° C.

In some embodiments, the compound of Formula (E-α-1) is produced by reacting a compound of Formula (D-α-1) with hydrogen and Pd/C for a reaction time between 4 hours and 13 hours, 6 hours and 13 hours, 7 hour and 13 hours, 8 hours and 12 hours, 9 hours and 12 hours, or 10 hours and 12 hours; or for less than 15 hours, less than 14 hours, less than 13 hours, less than 12 hours or less than 1 hours. In one variation, the reaction time is between 10 and 12 hours.

With reference again to FIG. 1, in some embodiments, the compound of Formula (G-α-1), or a salt thereof, or a hydrate of any of the foregoing, is produced by deprotecting the compound of Formula (E-α-1). In some variations, the compound of Formula (G-α-1) is:

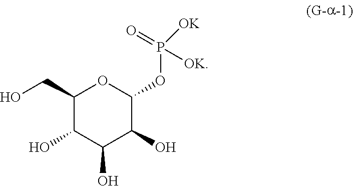

In some embodiments, the compound of Formula (G-α-1) is produced by reacting a compound of Formula (E-α-1) with a suitable base. In some embodiments, the base is an inorganic base. In some embodiments, the base is KOMe, NaOMe, LiOMe, ^{t}BuOK, ^{t}BuONa, ^{t}BuOLi, NaOEt, KOEt, or LiOEt. In one variation, the base is KOMe. It will be understood by those skilled in the art that the choice of base used in this step determines the specific salt form of the compound of Formula (G-α-1). For example, if NaOMe is used rather than KOMe, a sodium salt as depicted below will be obtained:

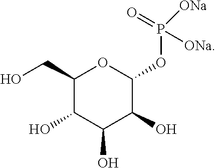

In some embodiments, the compound of Formula (G-α-1) is produced by reacting a compound of Formula (E-α-1) at a temperature between 10° C. and 30° C., between 15° C. and 30° C., or between 20° C. and 30° C. In one variation, the reaction is performed between 20° C. and 25° C.

In some embodiments, the compound of Formula (G-α-1) is produced by reacting a compound of Formula (E-α-1) for a reaction time between 10 hours and 30 hours, 15 hours and 30 hours, 20 hours and 30 hours, 20 hours and 25 hours, or 20 hours and 22 hours; or for less than 35 hours, less than 30 hours, less than 25 hours, or less than 22 hours. In one variation, the reaction time is between 20 and 22 hours.

### Compositions Comprising Alpha Isomer of M1P

In some aspects, provided is a composition produced according to the methods provided herein. In some embodiments, the composition comprises an alpha isomer of mannose-1-phosphate (M1P), or a salt thereof, or a hydrate of any of the foregoing, and the purity of the composition is at least 90%, at least 91%, at least 92%, at least 93%, at least 94%, at least 95%, at least 96%, at least 97%, at least 98%, or at least 99%. In one variation, the purity of the composition is at least 96%. In one variation of the foregoing, purity refers to the amount of the main component (in this instance, an alpha isomer of mannose-1-phosphate (M1P), or a salt thereof, or a hydrate of any of the foregoing) in a sample of the composition.

In some embodiments, the composition comprising an alpha isomer of M1P, or a salt thereof, or a hydrate of any of the foregoing, contains less than 10%, less than 9%, less than 8%, less than 7%, less than 6%, less than 5%, less than 4%, less than 3%, less than 2%, or less than 1% of a beta isomer of M1P, or a salt thereof, or a hydrate of any of the foregoing. In one variation, the composition contains less than 2% of a beta isomer of M1P, or a salt thereof, or a hydrate of any of the foregoing.

In some embodiments, the composition comprising an alpha isomer of M1P, or a salt thereof, or a hydrate of any of the foregoing, contains less than 10%, less than 9%, less than 8%, less than 7%, less than 6%, less than 5%, less than 4%, less than 3%, less than 2%, or less than 1% of

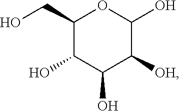 [SMILES: OC[C@H]1OC(O)[C@@H](O)[C@@H](O)[C@@H]1O]

or a salt thereof, or a hydrate of any of the foregoing. In one variation, the composition contains less than 1% of

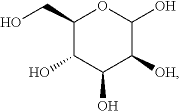 [SMILES: OC[C@H]1OC(O)[C@@H](O)[C@@H](O)[C@@H]1O]

or a salt thereof, or a hydrate of any of the foregoing.

In some embodiments, the composition comprising an alpha isomer of M1P, or a salt thereof, or a hydrate of any of the foregoing, contains less than 10%, less than 9%, less than 8%, less than 7%, less than 6%, less than 5%, less than 4%, less than 3%, less than 2%, or less than 1% of

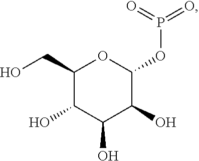 [SMILES: O=P(=O)O[C@H]1O[C@H](CO)[C@@H](O)[C@H](O)[C@@H]1O]

or a salt thereof, or a hydrate of any of the foregoing.

In one variation, the composition contains less than 1% of

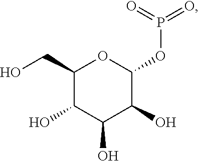 [SMILES: O=P(=O)O[C@H]1O[C@H](CO)[C@@H](O)[C@H](O)[C@@H]1O]

or a salt thereof, or a hydrate of any of the foregoing.

In some embodiments, the composition comprising an alpha isomer of M1P, or a salt thereof, or a hydrate of any of the foregoing, contains less than 10%, less than 9%, less than 8%, less than 7%, less than 6%, less than 5%, less than 4%, less than 3%, less than 2%, or less than 1% of

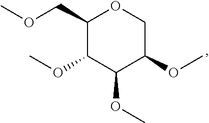

or a salt thereof, or a hydrate of any of the foregoing. In one variation, the composition contains less than 1% of

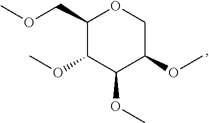

or a salt thereof, or a hydrate of any of the foregoing.

### Crystalline Form C and Methods of Producing Thereof

In other aspects, provided is a crystalline form of a potassium hydrate salt of M1P. Such crystalline form is referred to herein as “Form C”. Form C is a dipotassium salt. In some variations, Form C may be hydrated with between 2.5 and 3.5 water molecules per molecule of M1P. In one variation, Form C is specifically a dipotassium trihydrate salt having the structure:

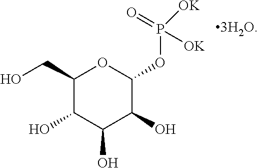

Overall, Form C was observed to be more stable than other identified polymorphs, including Forms A and B. Form C shows good crystallinity; exhibits physical and chemical stability, and is only slightly hygroscopic; has no form change in 80% RH at 25° C.; and shows good solubility of >160 mg/mL in pH 7.0 Tris buffer over 24 hours at 37° C.

In certain aspects, Form C of M1P can be produced from (i) a substantially pure alpha isomer of mannose-1-phosphate, or a salt thereof, or a hydrate of any of the foregoing, or (ii) crystalline Form A of a substantially pure alpha isomer of mannose-1-phosphate, or a salt thereof, or a hydrate of any of the foregoing, or (iii) crystalline Form B of a substantially pure alpha isomer of mannose-1-phosphate, or a salt thereof, or a hydrate of any of the foregoing, or any combination of (i)-(iii).

In some embodiments, the method of producing a crystalline Form C of M1P comprises: combining a starting composition comprising (i) substantially pure alpha isomer of mannose-1-phosphate, or a salt thereof, or a hydrate of any of the foregoing, or (ii) crystalline Form A of a substantially pure alpha isomer of mannose-1-phosphate, or a salt thereof, or a hydrate of any of the foregoing, or (iii) crystalline Form B of a substantially pure alpha isomer of mannose-1-phosphate, or a salt thereof, or a hydrate of any of the foregoing, or any combination of (i)-(iii), with a solvent comprising water and alcohol to produce a suspension, and stirring the suspension to produce a product composition comprising crystalline Form C of mannose-1-phosphate. In some embodiments, the starting composition comprises the alpha isomer of mannose-1-phosphate, or a salt thereof, or a hydrate of any of the foregoing, and has less than 10%, less than 9%, less than 8%, less than 7%, less than 6%, less than 5%, less than 4%, less than 3%, less than 2%, or less than 1% of the beta isomer of mannose-1-phosphate, or a salt thereof, or a hydrate of any of the foregoing. In some embodiments, the starting composition comprises a mixture of crystalline Form A and crystalline Form B of mannose-1-phosphate. In some embodiments, the starting composition comprises crystalline Form A of mannose-1-phosphate. In some embodiments, the starting composition comprises crystalline Form B of mannose-1-phosphate. In some variations, the solvent comprising water and alcohol comprises water and methanol in a ratio of between 5:1 and 1:10 v/v. In other variations, the suspension is allowed to equilibrate for a time between 0 weeks and 1 week, between 1 week and 2 weeks, or between 2 weeks and 3 weeks. In some embodiments, the suspension is allowed to equilibrate at a temperature between 0° C. and 5° C., between 5° C. and 10° C., between 10° C. and 20° C., between 20° C. and 30° C., or between 30° C. and 60° C.

In another aspect, provided is a crystalline Form C of M1P, produced according to any of the methods described herein. In yet another aspect, provided is a composition comprising a crystalline Form C of M1P.

In some embodiments, crystalline Form C of M1P is a substantially pure alpha isomer of a potassium trihydrate salt of mannose-1-phosphate. In some variations, Form C has less than 10%, less than 9%, less than 8%, less than 7%, less than 6%, less than 5%, less than 4%, less than 3%, less than 2%, or less than 1% of the corresponding beta isomer. In one variation, Form C has less than 1% of the corresponding beta isomer.

In some embodiments, Form C has an X-ray powder diffraction (XRPD) pattern comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or all peaks selected from 11.6, 13.4, 14.0, 14.6, 15.0, 16.1, 17.8, 19.0, 22.0, 22.4, 23.1, 23.2, 25.2, 25.8, 27.0, 27.6, 27.7, 28.5, 29.5, 30.1, 30.7, 32.2, 32.9, 34.5, 34.7, 35.1, 35.9, 36.6, 37.0, 37.8, 38.3, 39.0, and 39.7±0.2 degrees 2-theta.

In some embodiments, Form C has an X-ray powder diffraction (XRPD) pattern comprising at least 1, 2, 3, 4, 5, 6, 7, 8, 9, 10, 11, 12, 13, 14, or all peaks selected from 11.6, 14.6, 15.0, 16.1, 17.8, 22.0, 23.2, 25.2, 25.8, 26.9, 27.7, 29.5, 30.7, 32.2, and 35.1±0.2 degrees 2-theta.

In some embodiments, Form C has an X-ray powder diffraction (XRPD) pattern comprising at least 1, 2, 3, 4, 5, 6, 7 or all peaks selected from 11.6, 14.6, 17.8, 22.0, 23.2, 27.7, 29.5, and 30.7±0.2 degrees 2-theta. In some embodiments, Form C has an X-ray powder diffraction (XRPD) pattern comprising at least 1, 2, 3, 4, 5, or all peaks selected from 11.6, 14.6, 22.0, 27.7, 29.5, and 30.7±0.2 degrees 2-theta. In some embodiments, Form C has an X-ray powder diffraction (XRPD) pattern comprising at least 1, 2, 3, 4, 5, or all peaks selected from 11.6, 14.6, 17.8, 23.2, 27.7, and 30.7±0.2 degrees 2-theta. In one variation, Form C has an X-ray powder diffraction (XRPD) pattern comprising peaks selected from 11.6, 14.6, 17.8, 23.2, 27.7, and 30.7±0.2 degrees 2-theta. In another variation, Form C has an X-ray powder diffraction (XRPD) pattern comprising peaks selected from 11.6, 14.6, 17.8, 23.2, 27.7, and 30.7±0.1 degrees 2-theta.

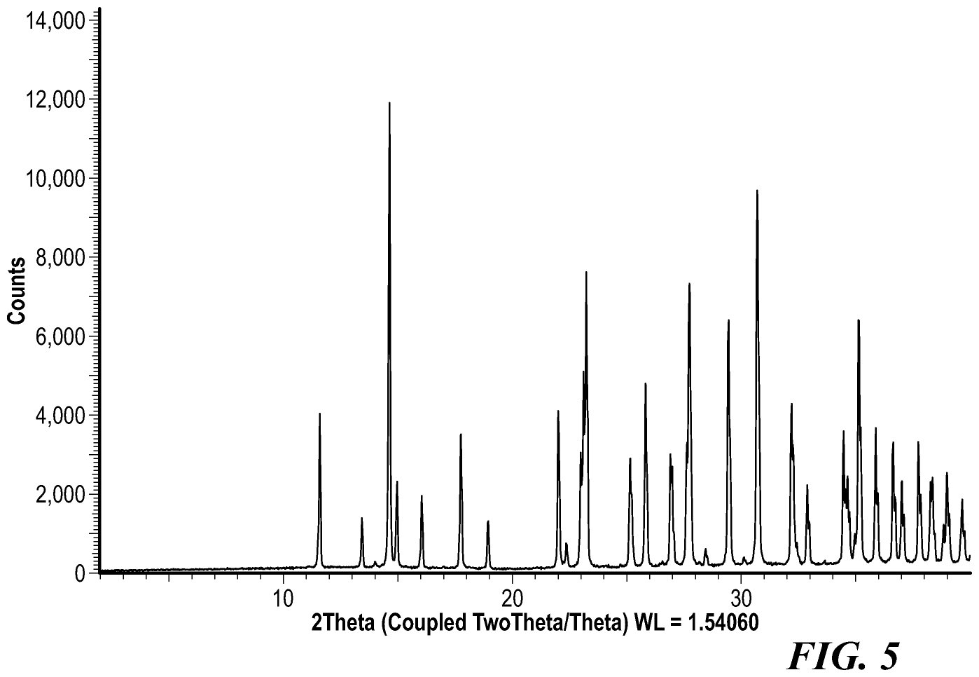

In one variation, Form C has an XRPD substantially as shown in FIG. 5.

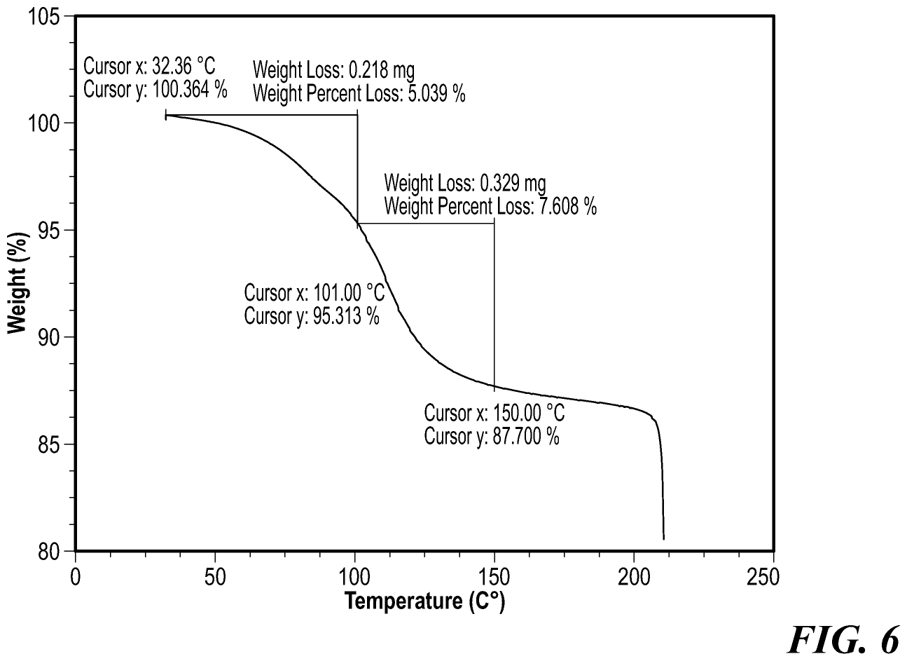

In another variation, Form C exhibits a TGA thermogram substantially similar to FIG. 6. In some embodiments, Form C exhibits a TGA thermogram with a weight loss of about 5% to 8% in the temperature range of 101-150° C.

In other embodiments, the crystalline Form C of M1P has an average particle size between 1 μm and 20 μm. In some embodiments, Form C, when compressed for 5 minutes under 5 MPa and/or under 10 MPa, does not undergo a form change, or does not undergo a substantial form change. In some embodiments, Form C, when ground, whether under dry conditions or wet conditions, does not undergo a form change, or does not undergo a substantial form change. In some embodiments, Form C, when stored for one week in an open container at about 40° C. and about 75% relative humidity, does not undergo a form change, or does not undergo a substantial form change. In some embodiments, Form C, when stored for one week in a closed container at about 60° C., does not undergo a form change, or does not undergo a substantial form change.

In some embodiments, Form C of M1P (including any of the compositions comprising Form C of M1P as produced according to the methods herein) is stable under one or more of the following conditions: when compressed for 5 minutes under 5 MPa and/or under 10 MPa; when ground, whether under dry conditions or wet conditions; when stored for one week in an open container at about 40° C. and about 75% relative humidity; and/or when stored for one week in a closed container at about 60° C.

In other embodiments, Form C of M1P (including any of the compositions comprising Form C of M1P as produced according to the methods herein) is stable when stored for up to about twelve months under one or more of the following conditions: at about −20° C.; at about 25° C. and about 60% relative humidity; at about 5° C.; or at about 40° C. and about 75% relative humidity.

In some variations of the foregoing, Form C is stable when its purity remains substantially unchanged over the storage period. In certain variations of the foregoing, Form C is stable when form change does not occur or substantially occur; and/or when any changes in purity over the storage period is less than 10%, less than 5%, or less than 1%, or not detectable. Any suitable methods may be used to characterize stability and purity, including for example by XRPD and/or chromatography.

In some aspects, provided is a composition comprising at least 95%, at least 96%, at least 97%, at least 98%, or at least 99% by weight of a crystalline Form C, which may be produced according to any of the methods described herein. In one variation, the composition comprises at least 98% by weight of a crystalline Form C. In some embodiments, the composition has less than 5%, less than 4%, less than 3%, less than 2%, less than 1%, less than 0.5%, or less than 0.1% of the corresponding beta isomer of M1P potassium salt having the structure:

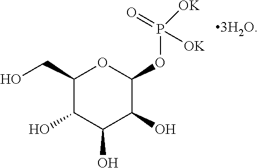

In some embodiments, the composition has less than 5%, less than 4%, less than 3%, less than 2%, less than 1%, less than 0.5%, or less than 0.1% of crystalline Form A and/or crystalline Form B of M1P. In one variation, less than 0.5% of the composition is Form A and/or Form B of mannose-1-phosphate.

In some embodiments, Form A has an X-ray powder diffraction (XRPD) pattern comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or all 38 peaks selected from 9.1, 12.7, 13.3, 14.8, 16.0, 16.4, 17.8, 18.2, 20.3, 20.6, 21.1, 21.5, 23.0, 23.3, 23.5, 24.4, 24.7, 25.2, 26.4, 27.1, 27.3, 28.1, 29.0, 29.7, 30.8, 31.3, 32.0, 33.0, 33.7, 34.4, 35.1, 36.3, 37.3, 37.7, 38.0, 38.6, 39.1, and 39.4±0.2 and ±0.2 degrees 2-theta.

In some embodiments, Form A has an X-ray powder diffraction (XRPD) pattern comprising at least 1, 2, 3, 4, 5, 6, 7, 8, 9, 10, 11, 12, 13, 14, 15, or all 16 peaks selected from 9.1, 13.3, 16.0, 17.8, 20.3, 21.1, 21.5, 23.5, 25.2, 26.4, 27.1, 28.1, 29.0, 31.3, 32.0, and 33.0±0.2 degrees 2-theta.

In some embodiments, Form A has an X-ray powder diffraction (XRPD) pattern comprising at least 1, 2, 3, 4, 5, or all 6 peaks selected from 20.3, 21.1, 23.5, 27.1, 28.1, and 29.0±0.2 degrees 2-theta.

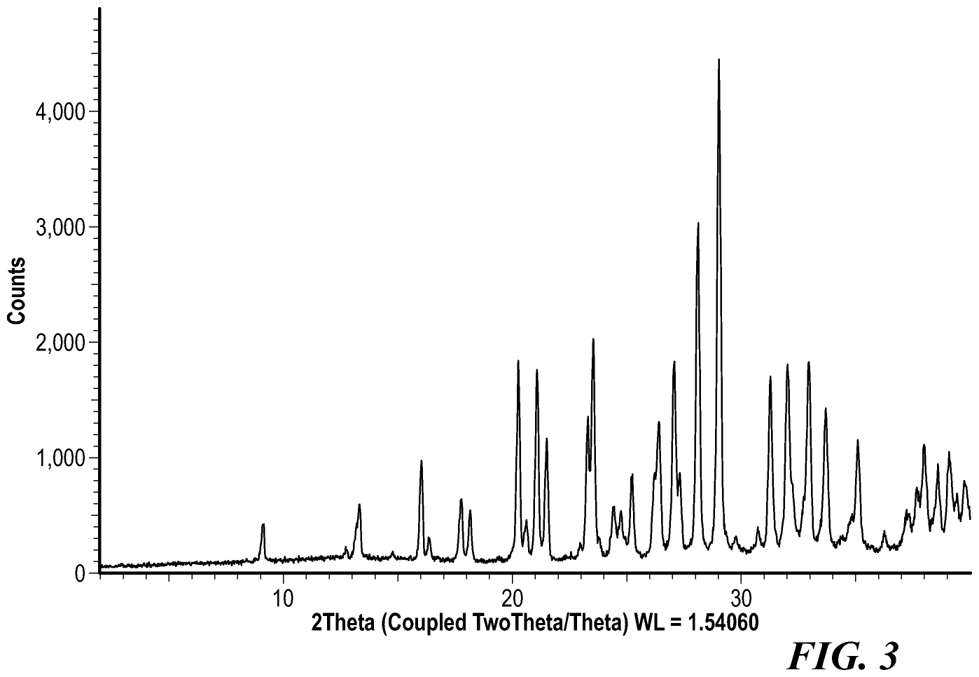

In some variations, crystalline Form A has an XRPD substantially as shown in FIG. 3.

In some embodiments, Form B has an X-ray powder diffraction (XRPD) pattern comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or all 43 peaks selected from 4.2, 8.3, 12.2, 13.5, 14.2, 15.7, 16.1, 16.4, 16.7, 17.2, 17.6, 19.2, 19.8, 20.6, 21.4, 22.6, 23.2, 23.9, 24.5, 25.8, 26.2, 26.6, 27.5, 28.0, 28.6, 28.9, 29.3, 29.8, 30.6, 30.9, 31.3, 32.3, 32.9, 33.4, 34.0, 34.5, 34.8, 35.3, 36.7, 37.3, 37.6, 39.3, and 39.9±0.2 degrees 2-theta.

In some embodiments, Form B has an X-ray powder diffraction (XRPD) pattern comprising at least 1, 2, 3, 4, 5, 6, 7, 8, 9, 10, 11, 12, 13, 14, 15, 16, 17, 18, 19, or all 20 peaks selected from 8.3, 14.2, 16.1, 16.4, 17.2, 19.8, 21.4, 23.2, 23.9, 24.5, 26.2, 26.6, 27.5, 28.6, 29.3, 29.8, 30.6, 32.3, 32.9 and 34.8±0.2 degrees 2-theta.

In some embodiments, Form B has an X-ray powder diffraction (XRPD) pattern comprising at least 1, 2, 3, 4, 5, or all 6 peaks selected from 8.3, 14.2, 19.8, 21.4, 23.9, and 27.5±0.2 degrees 2-theta.

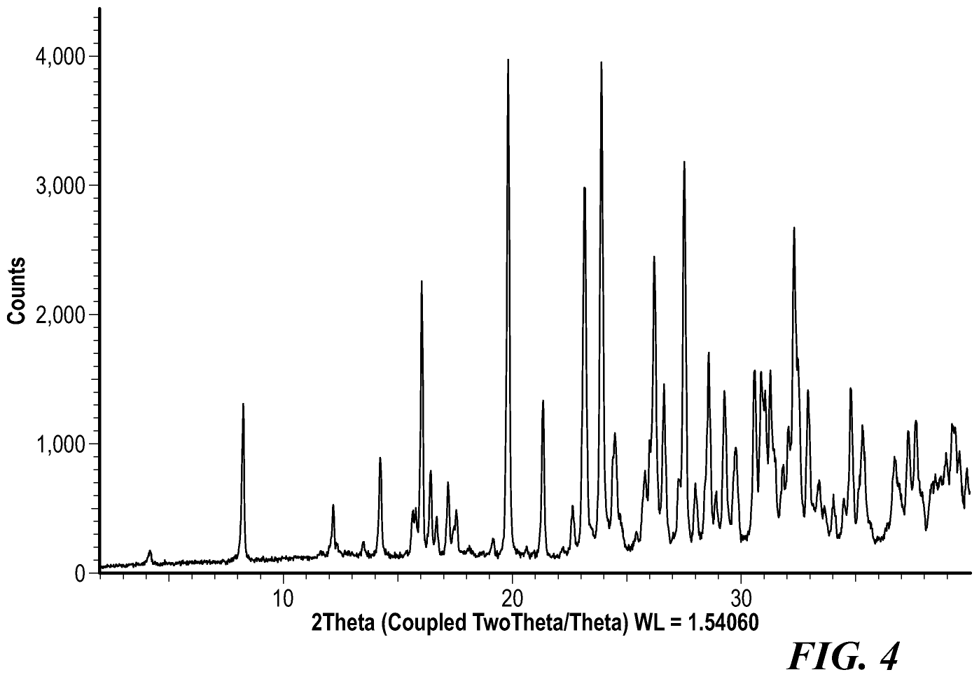

In some variations, crystalline Form B has an XRPD substantially as shown in FIG. 4.

### Pharmaceutical Compositions

In some aspects, provided is a composition as described herein, further comprising at least one pharmaceutically acceptable carriers, excipients, or stabilizers. In some embodiments, pharmaceutically acceptable carriers, excipients, or stabilizers are nontoxic to the cell or subject being exposed thereto at the dosages and concentrations employed. Often the physiologically acceptable carrier is an aqueous pH buffered solution.

In one aspect, provided is a lipid particle that contain any of the compositions described herein (including, e.g., compositions comprising an alpha isomer of mannose-1-phosphate (M1P), or a salt thereof, or a hydrate of any of the foregoing), encapsulated within the lipid particle. In some embodiments, the lipid particle refers to particles formed by lipids in an aqueous solution. Suitable examples of lipid particles include, without limitation, liposomes, micelles, solid lipid nanoparticles, niosome, lipospheres, emulsomes, and emulsions.

In some variations, encapsulation in a lipid particle refers to a lipid particle that provides an active agent or therapeutic agent, such as any of the compositions comprising an alpha isomer of mannose-1-phosphate (M1P), or a salt thereof, or a hydrate of any of the foregoing, with full encapsulation, partial encapsulation, or both. In some variations, at least a portion of the any of the compositions comprising an alpha isomer of mannose-1-phosphate (M1P), or a salt thereof, or a hydrate of any of the foregoing, may be encapsulated by a lipid particle and localized within the core of a lipid particle and/or within the inner surface (e.g., the membrane) of a lipid particle. Alternatively, in other variations, the entire compositions comprising an alpha isomer of mannose-1-phosphate (M1P), or a salt thereof, or a hydrate of any of the foregoing, may be encapsulated by a lipid particle and localized within the core of a lipid particle and/or within the inner surface (e.g., the membrane) of a lipid particle.

Any lipid particle known in the art suitable for delivering an encapsulated carbohydrate of the present disclosure to the interior of a cell may be used. Examples of suitable lipid particles include, without limitation, liposomes, micelles, solid lipid nanoparticles, and niosomes.

### Liposomes

In some embodiments, a lipid particle of the present disclosure may be a liposome. As used herein, a liposome refers to a vesicle composed of a lamellar phase lipid bilayer. Any suitable liposome known in the art may be used. In some embodiments, the liposome has a lamellar nanostructure. In some variations, lamellar nanostructure refers to a nanostructure, such as a lipid particle, that includes parallel amphiphilic bilayers separated by a lumen.

Liposomes of the present disclosure may be prepared by any suitable method known in the art and disclosed herein. Examples of suitable methods for preparing liposomes include, without limitation, disrupting biological membranes, such as by mechanical dispersion including sonication, thin-film hydration, emulsions, french pressure cell, extrusion, and reconstitution of dried vesicles; solvent dispersion including ethanol injection, ether injection, double emulsion, reverse phase, and vaporization; and detergent removal methods.

In certain embodiments, the liposome is a stealth liposome that may be immunotolerant. In some variations, a stealth liposome refers to liposomes that are capable of avoiding detection by a subject's immune system. As such, a stealth liposome may be immunotolerant.

In one aspect, provided herein is a composition comprising: (i) a liposome comprising one or more phospholipid; and (ii) alpha isomer of mannose-1-phosphate, or a salt thereof, or a hydrate of any of the foregoing, encapsulated in the liposome. In one embodiment, provided herein is a composition comprising: (i) a liposome comprising one or more phospholipids conjugated to polyethylene glycol (PEG); and (ii) alpha isomer of mannose-1-phosphate, or a salt thereof, or a hydrate of any of the foregoing, encapsulated in the liposome.

In some embodiments, provided is a composition, comprising: liposomes having a lipid membrane enclosing an intraliposomal compartment, wherein the liposomes encapsulate in the intraliposomal compartment alpha isomer of mannose-1-phosphate, or a salt thereof, or a hydrate of any of the foregoing; intraliposomal buffer; extraliposomal buffer; and optionally a radical scavenging antioxidant.

In some embodiments, provided herein is a composition, comprising: liposomes having a lipid membrane enclosing an intraliposomal compartment, wherein the liposomes encapsulate in the intraliposomal compartment an alpha isomer of mannose-1-phosphate, or a salt thereof, or a hydrate of any of the foregoing; intraliposomal buffer comprising a buffer salt and optionally acid, wherein the pKa of the buffer salt is between 6 to 8.5; extraliposomal buffer comprising a buffer salt and a tonicity modifier, wherein the pKa of the buffer salt is between 6 to 8.5; and optionally a radical scavenging antioxidant.

In some variations, the lipid membrane comprises: (a) at least one phospholipid having an ethanolamine head group and at least one unsaturated fatty acid tail; (b) at least one phospholipid having a choline group and at least one unsaturated fatty acid tail; and (c) at least one phospholipid having an ethanolamine head group and at least one saturated fatty acid tail. In one variation, the lipid membrane comprises: (a) at least one phospholipid having an ethanolamine head group and at least one unsaturated fatty acid tail; (b) at least one phospholipid having a choline group and at least one unsaturated fatty acid tail; and (c) at least one phospholipid having an ethanolamine head group and at least one saturated fatty acid tail, conjugated to polyethylene glycol (PEG).

In some variations of the compositions comprising liposomes and an alpha isomer of M1P, the alpha isomer of M1P is produced according to any of the methods provided herein. In some embodiments, the compositions contain less than 5%, less than 4%, less than 3%, less than 2%, less than 1%, less than 0.5%, or less than 0.1% of the beta isomer of mannose-1-phosphate, or a salt thereof, or a hydrate of any of the foregoing.

In some variations of the compositions comprising liposomes and an alpha isomer of M1P, the alpha isomer of mannose-1-phosphate potassium hydrate salt is Form C. In other embodiments, less than 0.5% of the composition is Form A and/or Form B of mannose-1-phosphate.

### Micelles

In some embodiments, a lipid particle of the present disclosure may be a micelle. In some variations, a micelle refers to an aggregate of surfactant molecules (e.g., soaps, detergents, fatty acids, lipids, phospholipids, etc.) that are dispersed in a liquid colloid. A micelle in aqueous solution may form an aggregate with hydrophilic head regions in contact with surrounding solvent, sequestering the hydrophobic tail regions in the interior of the micelle. Any suitable micelle known in the art may be used. In some embodiments, micelles may be spherical. Micelles of the present disclosure may be prepared by any suitable method known in the art. Examples of suitable methods for preparing micelles include, without limitation, direct dissolution, and direct or microemulsification dialysis, which may encompass preparation by detergent or water-miscible solvent removal methods.

### Solid Lipid Nanoparticles

In some embodiments, a lipid particle of the present disclosure may be a solid lipid nanoparticle. In some variations, a solid lipid nanoparticle (SLN) refers to lipid in water emulsions composed of lipids that are generally solid at temperatures of at least 50° C., and typically contain a solid lipid core matrix that can solubilize lipophilic molecules. In some variations, solid lipid nanoparticles have a diameter in the range of 10 to 1000 nanometers. Solid lipid nanoparticles may protect incorporated active compounds, such as carbohydrates of the present disclosure, against chemical degradation and can also demonstrate flexibility in modulating the release of such compounds. The lipid core of solid lipid nanoparticles may be stabilized by surfactants (e.g., emulsifiers). The lipid may typically include triglycerides, diglycerides, monoglycerides, fatty acids, steroids, and/or waxes. Any suitable solid lipid nanoparticle known in the art may be used. Solid lipid nanoparticles of the present disclosure may be prepared by any suitable method known in the art. Examples of suitable methods for preparing solid lipid nanoparticles include, without limitation, microemulsification, high-pressure homogenization, precipitation, and film ultrasound dispersion.

### Niosomes

In some embodiments, a lipid particle of the present disclosure may be a niosome. In some variations, a niosome refers to a vesicular structure formed of a bilayer of non-ionic surfactant molecules and that contains an aqueous core. Niosomes are structurally similar to liposomes in having a lamellar structure, however, the materials used to prepare niosomes make them more stable against hydrolytic degradation. Examples of suitable noisome preparation materials include, without limitation, sterols and one or more non-ionic surfactants. Any suitable niosome known in the art may be used. Niosomes of the present disclosure may be prepared by any suitable method known in the art. Examples of suitable methods for preparing micelles include, without limitation, ether injection, agitation, bubble method, reverse phase evaporation, sonication, multiple membrane extrusion, and microfluidigation.

### Formulations

Examples of suitable formulations incorporating the compositions described herein may include, without limitation, solutions, injections, inhalants, microspheres, aerosols, gels, ointments, creams, lotions, powders, dry vescicular powders, tablets, and capsules. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include, without limitation, other carriers or non-toxic, nontherapeutic, nonimmunogenic stabilizers, and excipients. The compositions can also include additional substances to approximate physiological conditions, such as pH adjusting and buffering agents, toxicity adjusting agents, wetting agents and detergents. A pharmaceutical composition of the present disclosure can also include any of a variety of stabilizing agents, such as an antioxidant for example.

In some variations, the pharmaceutical compositions described herein may be formulated for injection into a subject in need thereof, such as a human in need thereof. Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

In some variations, the composition provided herein is formulated for intravenous administration.

In some variations of the compositions comprising lipid particle (including, e.g., liposomes) and an alpha isomer of M1P, after the composition is stored at controlled room temperature, the purity of the alpha isomer of mannose-1-phosphate in the composition is at least about 94%, at least about 95%, at least about 96%, at least about 97%, or at least about 98%. In one variation, the purity of the alpha isomer of mannose-1-phosphate in the composition is at least about 98%.

In some variations of the compositions comprising lipid particle (including, e.g., liposomes) and an alpha isomer of M1P, after the composition is stored at ambient temperature, the purity of the alpha isomer of mannose-1-phosphate in the composition is at least about 91%, at least about 92%, at least about 93%, at least about 94%, or at least about 95%. In one variation, the purity of the alpha isomer of mannose-1-phosphate in the composition is at least about 95%.

Suitable phospholipids, buffers and radical scavenging antioxidants may be used the pharmaceutical compositions, and are described in further detail below.

### Phospholipids

In some embodiments, the lipid membrane comprises: (a) at least one phospholipid having an ethanolamine head group and at least one unsaturated fatty acid tail; (b) at least one phospholipid having a choline group and at least one unsaturated fatty acid tail; and (c) at least one phospholipid having an ethanolamine head group and at least one saturated fatty acid tail.

In one embodiment, the lipid membrane comprises: (a) at least one phospholipid having an ethanolamine head group and at least one unsaturated fatty acid tail; (b) at least one phospholipid having a choline group and at least one unsaturated fatty acid tail; and (c) at least one phospholipid having an ethanolamine head group and at least one saturated fatty acid tail, conjugated to polyethylene glycol (PEG).

In certain embodiments with respect the phospholipid having an ethanolamine head group in (a) of the lipid membrane, the unsaturated fatty acid tail independently comprises at least one C10-28 carbon chain. In certain embodiments, the unsaturated fatty acid tail is an optionally substituted C10-28 alkenyl or an optionally substituted C10-28 alkynyl. In some embodiments, each of the fatty acid chains is an unsubstituted C10-28 alkenyl. In some embodiments, the alkenyl is linear or branched. In some embodiments, each of the fatty acid chains has one or more double bonds. In some embodiments, each double bond has cis configuration. In some embodiments, each double bond has trans configuration. In some embodiments, each of the fatty acid chains is a C10-28 alkenyl substituted by a substituent selected form the group consisting of acyl, hydroxyl, cycloalkyl, alkoxy, acyloxy, amino, aminoacyl, nitro, halo, thiol, thioalkyl, alkyl, alkenyl, alkynyl and heterocyclyl. In some variations, the phospholipid having an ethanolamine head group in (a) of the lipid membrane has oleoyl tail groups.

In some embodiments, the phospholipid having an ethanolamine head group and at least one unsaturated fatty acid tail is 1,2-dioleoyl-sn-glycero-3-phosphoethanolamine (DOPE) or a salt thereof. In one variation, the phospholipid having an ethanolamine head group and at least one unsaturated fatty acid tail is:

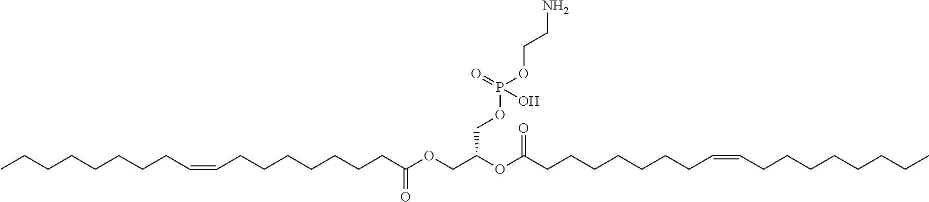 [SMILES: CCCCCCCC/C=C\CCCCCCCC(=O)OC[C@H](COP(=O)(O)OCCN)OC(=O)CCCCCCC/C=C\CCCCCCCC]

or a salt thereof.

In certain embodiments with respect to the phospholipid having a choline group in (b) of the lipid membrane, the unsaturated fatty acid tail independently comprises at least one C10-28 carbon chain. In certain embodiments, the unsaturated fatty acid tail is an unsubstituted C10-28 alkenyl. In some embodiments, the alkenyl is linear or branched. In some embodiments, the unsaturated fatty acid tail has one or more double bonds. In some embodiments, each double bond has cis configuration. In some embodiments, each double bond has trans configuration. In some embodiments, the unsaturated fatty acid tail is a C10-28 alkenyl substituted by a substituent selected form the group consisting of acyl, hydroxyl, cycloalkyl, alkoxy, acyloxy, amino, aminoacyl, nitro, halo, thiol, thioalkyl, alkyl, alkenyl, alkynyl and heterocyclyl. In some variations, the phospholipid having choline group in (b) of the lipid membrane has oleoyl tail groups.

In some variations, the phospholipid having a choline group and at least one unsaturated fatty acid tail is 1,2-dioleoyl-sn-glycero-3-phosphocholine (DOPC), or a salt thereof. In some variations, the phospholipid having a choline group and at least one unsaturated fatty acid tail is:

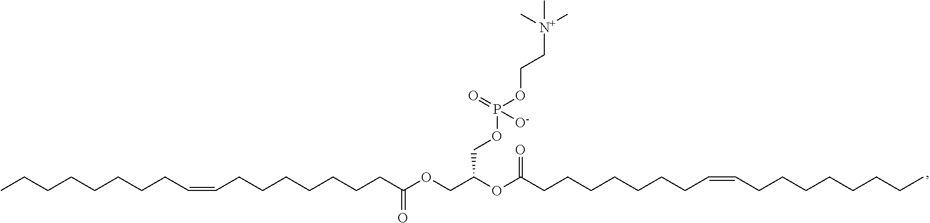 [SMILES: CCCCCCCC/C=C\CCCCCCCC(=O)OC[C@H](COP(=O)([O-])OCC[N+](C)(C)C)OC(=O)CCCCCCC/C=C\CCCCCCCC]

or a salt thereof.

In some embodiments with respect to the phospholipid having an ethanolamine head group in (c) of the lipid membrane, the saturated fatty acid tail independently comprises at least one C4-28 carbon chain. In certain embodiments, the saturated fatty acid tail is an optionally substituted alkyl. In some embodiments, the saturated fatty acid tail is an unsubstituted C4-28 alkyl. In some embodiments, the saturated fatty acid tail is a C4-28 alkyl substituted by a substituent selected form the group consisting of acyl, hydroxyl, cycloalkyl, alkoxy, acyloxy, amino, aminoacyl, nitro, halo, thiol, thioalkyl, alkyl, alkenyl, alkynyl and heterocyclyl. In some variations, the phospholipid having an ethanolamine head group in (c) of the lipid membrane has stearoyl tail groups.

In some variations, the phospholipid having an ethanolamine head group and at least one saturated fatty acid tail is 1,2-distearoyl-sn-glycero-3-phosphoethanolamine (DSPE), or a salt thereof. In some variations, the phospholipid having an ethanolamine head group and at least one saturated fatty acid tail is:

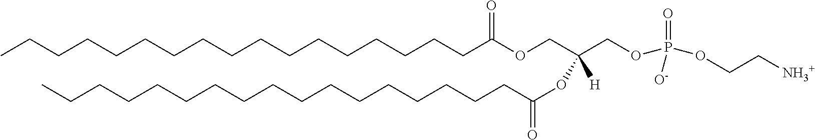 [SMILES: CCCCCCCCCCCCCCCCCC(=O)OC[C@H](COP(=O)([O-])OCC[NH3+])OC(=O)CCCCCCCCCCCCCCCCC]

or a salt thereof.

In certain variations, the phospholipid conjugated to PEG is DSPE or a salt thereof. In some embodiments, PEGylated phospholipid is DSPE-PEG. In some embodiments, PEGylated phospholipid is DSPE-PEG₂₀₀₀. In some embodiments, the DSPE-PEG is further conjugated to a carbohydrate. In certain embodiments, the DSPE-PEG is further conjugated to a monosaccharide. In some embodiments, the DSPE-PEG is further conjugated to a galactose moiety. In certain embodiments, the DSPE-PEG-galactose has the following structure:

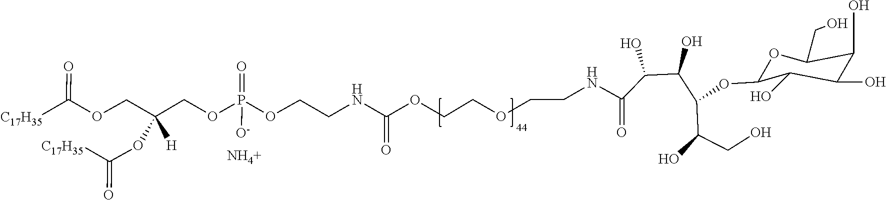

or a salt thereof.

In one embodiment, the lipid membrane comprises DOPC, DOPE and DSPE conjugated to PEG. In one variation, the lipid membrane comprises DOPE:DOPC:DSPE-PEG2000 at a ratio between 58.2:38.8:3 and 30:67:3.

In certain variations, PEG is present in the composition at a concentration that ranges from about 0.5 molar percent to about 20 molar percent. In some embodiments, PEG is present in the composition at a concentration of about 0.5 molar percent, about 1 molar percent, about 2 molar percent, about 3 molar percent, about 4 molar percent, about 5 molar percent, about 6 molar percent, about 7 molar percent, about 8 molar percent, about 9 molar percent, about 10 molar percent, about 11 molar percent, about 12 molar percent, about 13 molar percent, about 14 molar percent, about 15 molar percent, about 16 molar percent, about 17 molar percent, about 18 molar percent, about 19 molar percent, or about 20 molar percent. In some embodiments, PEG is present in the composition at a concentration of at least about 0.5 molar percent, at least about 1 molar percent, at least about 2 molar percent, at least about 3 molar percent, at least about 4 molar percent, at least about 5 molar percent, at least about 6 molar percent, at least about 7 molar percent, at least about 8 molar percent, at least about 9 molar percent, at least about 10 molar percent, at least about 11 molar percent, at least about 12 molar percent, at least about 13 molar percent, at least about 14 molar percent, at least about 15 molar percent, at least about 16 molar percent, at least about 17 molar percent, at least about 18 molar percent, at least about 19 molar percent, or at least about 20 molar percent; or between 0.5 molar percent and 50 molar percent, between 0.5 molar percent and 40 molar percent, between 0.5 molar percent and 30 molar percent, or between 0.5 molar percent and 20 molar percent.

In certain variations, PEG is present in the composition at a molecular weight that ranges from about 200 Da to about 40,000 Da. In some embodiments, PEG is present in the composition at a molecular weight that ranges from about 200 Da to about 10,000 Da. In some embodiments, PEG is present in the composition at a molecular weight of about 200 Da, about 300 Da, about 400 Da, about 500 Da, about 600 Da, about 700 Da, about 800 Da, about 900 Da, about 1,000 Da, about 1,500 Da, about 2,000 Da, about 2,500 Da, about 3,000 Da, about 3,500 Da, about 4,000 Da, about 4,500 Da, about 5,000 Da, about 5,500 Da, about 6,000 Da, about 6,500 Da, about 7,000 Da, about 7,500 Da, about 8,000 Da, about 8,500 Da, about 9,000 Da, about 9,500 Da, or about 10,000 Da; or between about 200 Da and about 10,000 Da.

In one aspect, provided is a pharmaceutical composition comprising: a liposome comprising: i) 1,2-dioleoyl-sn-glycero-3-phosphoethanolamine (DOPE) or a salt thereof; ii) one or more phospholipids having a polar head group comprising glycerol, choline, phosphate or serine, and two unsaturated fatty acid tails, wherein each unsaturated fatty acid tail comprises a C10-28 alkenyl chain; and iii) polyethylene glycol (PEG) conjugated to at least one phospholipid; and any of the compositions described herein (including, e.g., compositions comprising an alpha isomer of mannose-1-phosphate (M1P), or a salt thereof, or a hydrate of any of the foregoing) encapsulated in the liposome. In some embodiments, the fatty acid comprising the polar head group is choline. In some embodiments, the one or more phospholipid having a polar head group is: 1,2-dioleoyl-sn-glycero-3-phosphocholine (DOPC); 1,2-dioleoyl-sn-glycero-3-phosphoglycerol (DOPG); or 1,2-dioleoyl-sn-glycero-3-phospho-L-serine (DOPS), or a salt thereof, or any combination of the foregoing. In certain variations, the one or more phospholipids having a polar head group comprise 1,2-dioleoyl-sn-glycero-3-phosphocholine (DOPC) or a salt thereof. In certain variations, the phospholipid conjugated to the PEG is 1,2-distearoyl-sn-glycero-3-phosphoethanolamine (DSPE). In one variation, component (iii) of the liposome is DSPE-PEG2000. In another variation, the liposome does not comprise cholesterol or a cholesterol ester. In certain variations, the DOPE is present in the composition from about 15 to about 30 molar percent. In another variation, the one or more phospholipid having a polar head group is DOPC, wherein the DOPC is present in the composition from about 35 to about 75 molar percent. In yet another variation, the one or more phospholipid having a polar head group is DOPC, wherein the DOPC is present in the composition from about 60 to about 65 molar percent. In certain embodiments, the DOPE is present in the composition from about 25 to about 30 molar percent. In yet other embodiments, the one or more phospholipid having a polar head group is DOPC, wherein the DOPC is present in the composition from about 35 to about 75 molar percent. In other embodiments, the one or more phospholipid having a polar head group is DOPC, wherein the DOPC is present in the composition from about 60 to about 65 molar percent. In some variations, the DOPE is present in the composition from about 30 to about 60 molar percent. In other variations, the one or more phospholipid having a polar head group is DOPC, wherein the DOPC is present in the composition from about 35 to about 75 molar percent. In yet other variations, the one or more phospholipid having a polar head group is DOPC, wherein the DOPC is present in the composition from about 60 to about 65 molar percent. In one variation, the average particle size of the liposomes in the composition is about 0.1 microns.

### Buffers

In some embodiments, the intraliposomal buffer comprises a buffer salt, and optionally acid. In some variations, acid may be added to the intraliposomal buffer to maintain a neutral pH.

In certain embodiments, the pKa of the buffer salt is between 6 to 8.5. In one embodiments, the intraliposomal buffer is in a physiological pH range. In one embodiment, the intraliposomal buffer is in a pH range between 7.35 to 6.45. In one variation, the buffer salt is tris(hydroxymethyl)aminomethane (Tris). In other variations, the intraliposomal buffer comprises bicarbonate, Tris, or HEPES, or any combination thereof.

In some embodiments, the extraliposomal buffer comprising a buffer salt and a tonicity modifier. In some variations, the pKa of the buffer salt is between 6 to 8.5. In one embodiments, the extraliposomal buffer is in a physiological pH range. In one variation, the buffer salt is tris(hydroxymethyl)aminomethane (Tris). In other variations, the extraliposomal buffer comprises bicarbonate, Tris, or HEPES, or any combination thereof.

In some variations, the tonicity modifier comprises sugar or saline, or a combination thereof. Suitable sugars that may be used for tonicity include, for example, sucrose and dextrose.

In other variations, the tonicity modifier is an ionic tonicity modifier. In one variation, the tonicity modifier comprises saline.

In some embodiments, the M1P and Tris, and optionally an acid, are present in the composition at a ratio suitable to maintain a neutral pH.

In one variation, the intraliposomal buffer comprises about 50 mM of Tris; and the extraliposomal buffer comprises 15 mM of Tris and at least 145 mM of saline.

### Radical Scavenging Antioxidants

Any suitable radical scavenging antioxidants may be used in the liposomal compositions provided herein. In some embodiments, the radical scavenging antioxidant is butylated hydroyanisole (BHA), butylated hydroxytoluene (BHT), or alpha tocopherol. In one variation, the radical scavenging antioxidant is BHT. In some variations, any suitable combinations of radical scavenging antioxidants may also be employed.

### Properties of the Pharmaceutical Compositions

The pharmaceutical compositions described herein are optimized for delivery of M1P to the cell interior, for treating diseases and disorders such as congenital disorders of glycosylation (CDG).

In some embodiments, the pharmaceutical composition has a drug-to-lipid (D/L) ratio of at least 0.1. In some embodiments, the composition minimizes both lipid degradation and liposomal agglomeration. In some variations, the lipid degradation of the composition is less than 10%, less than 9%, less than 8%, less than 7%, less than 6%, less than 5%, less than 4%, less than 3%, less than 2%, or less than 1%.

In some embodiments, the pharmaceutical composition has less than 10%, less than 9%, less than 8%, less than 7%, less than 6%, less than 5%, less than 4%, less than 3%, less than 2%, or less than 1% of total lipid impurities.

In some embodiments, the pharmaceutical composition maintains a pH range between 6.5 and 7, or between 7.35 and 7.45. In certain embodiments, the pharmaceutical composition maintains a physiological pH range.

In some embodiments, the pharmaceutical composition has a Z-average between 80 nm and 130 nm, between 80 nm and 120 nm, between 80 nm and 110 nm, between 80 nm and 100 nm, between 90 nm and 130 nm, between 90 nm and 120 nm, between 90 nm and 110 nm, or between 90 nm and 100 nm.

In some embodiments, the composition has a polydispersity index of less than 0.1.

In some embodiments, no free M1P is detected in the composition.

In some embodiments, the pharmaceutical composition has an encapsulation efficiency of at least 80%, at least 85%, at least 90%, at least 95%, or at least 99%.

In some embodiments, the pharmaceutical composition has a mean osmolality between 290 mOsm/kg and 320 mOsm/kg.

The compositions provided herein may have any one or more of the properties described above. For example, in some variations, the composition provided herein has all of the following properties: (i) a lipid degradation of less than 10%; (ii) a Z-average between 80 nm and 130 nm; (iii) an encapsulation efficiency of at least 80%; and (iv) a pH range between 6.5 and 7.

Stability of the compositions provided herein, as characterized based on for example lipid degradation, total lipid impurities, pH, Z-average, PDI, free M1P, encapsulation efficiency, and osmolality, may be measured over a time period over a range of temperatures, such as 5° C., 25° C. and 40° C. In some variations, the time period is 1-3 months. In other variations, the time period is at least 6 months, at least 1 year, or at least 2 years.

### Pharmaceutical Dosages

Pharmaceutical compositions described herein may be used in accord with known methods, such as oral administration, intravenous administration as a bolus or by continuous infusion over a period of time, by intramuscular, intraperitoneal, intracerobrospinal, intracranial, intraspinal, subcutaneous, intra-articular, intrasynovial, intrathecal, oral, topical, or inhalation routes.

Dosages and desired concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the compositions of the present disclosure, dosage amounts may vary from 10 ng/kg up to 100 mg/kg of a subject's body weight per day.

Administration of a composition of the present disclosure can be continuous or intermittent, depending, for example, on the recipient's physiological condition, whether the purpose of the administration is therapeutic or prophylactic, and other factors known to skilled practitioners.

It is within the scope of the present disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

Thus, in some variations, the compositions provided herein may be chronically or intermittently administered to a subject (including, for example, a human) in need thereof. “Chronic” administration refers to administration of the medicament(s) in a continuous as opposed to acute mode, so as to maintain the initial therapeutic effect (activity) for an extended period of time. “Intermittent” administration refers to treatment that is not consecutively done without interruption, but rather is cyclic in nature.

### Therapeutic Uses

The present disclosure provides compositions that are capable of delivering the carbohydrate into the interior of a cell. These compositions are useful for delivering phosphorylated carbohydrates of the present disclosure to a subject in need of such carbohydrates.

In some embodiments, the subject is a mammal. In one embodiment, the subject is a human. In some variations, the subject may be at risk. For example, in one variation, the subject is an at risk human. A subject at risk of developing a particular disease, disorder, or condition, such as a congenital disorder of glycosylation, may or may not have detectable disease or symptoms of disease, and may or may not have displayed detectable disease or symptoms of disease prior to the treatment methods described herein. In certain variations, an individual “at risk” is an individual having risk factors, which are measurable parameters that correlate with development of a particular disease, disorder, or condition, as known in the art. A subject having one or more of these risk factors has a higher probability of developing a particular disease, disorder, or condition such as a congenital disorder of glycosylation, than a subject without one or more of these risk factors.

In some embodiments, congenital disorders of glycosylation (CDG) is a group of genetic disorders that result in errors of metabolism in which glycosylation of a variety of tissue proteins and/or lipids is deficient or defective. Congenital disorders of glycosylation may also be known as CDG syndromes. CDG syndromes may often cause serious, occasionally fatal, malfunction of several different organ systems, such as the nervous system, brain, muscles, and intestines, in affected infants. Manifestations of CDG syndromes may range from severe developmental delay and hypotonia beginning in infancy, to hypoglycemia and protein-losing enteropathy with normal development. Developmental delay can be a common initial indication for a CDG diagnosis. One of the most common subtype of CDG syndromes is CDG-Ia (also known as PMM2-CDG) where the genetic defect leads to the loss of phosphomannomutase 2, which is the enzyme responsible for the conversion of mannose-6-phosphate into mannose-1-phosphate.

CDG syndromes may be classified as type I (CDG-I) and type II (CDG-II). Such classification may depend on the nature and location of the biochemical defect in the metabolic pathway relative to the action of oligosaccharyltransferase. Methods for screening for CDG subtype may include the analysis of transferrin glycosylation status by, for example, isoelectric focusing or ESI-MS. CDG type I include, for example, Ia (PMM2-CDG), Tb (MPI-CDG), Ic (ALG6-CDG), Id (ALG3-CDG), Ie (DPM1-CDG), If (MPDU1-CDG), Ig (ALG12-CDG), Ih (ALG8-CDG), Ii (ALG2-CDG), Ij (DPAGT1-CDG), Ik (ALG1-CDG), 1L (ALG9-CDG), Im (DOLK-CDG), In (RFT1-CDG), Io (DPM3-CDG), Ip (ALG11-CDG), Iq (SRD5A3-CDG), Ir (DDOST-CDG), DPM2-CDG, TUSC3-CDG, MAGT1-CDG, DHDDS-CDG, and I/IIx. CDG type II include, for example, IIa (MGAT2-CDG), IIb (GCS1-CDG), IIc (SLC335C1-CDG), IId (B4GALT1-CDG), IIe (COG7-CDG), IIf (SLC35A1-CDG), IIg (COG1-CDG), IIh (COG8-CDG), IIi (COG5-CDG), IIj (COG4-CDG), IIL (COG6-CDG), ATP6V0A2-CDG, MAN1B1-CDG, and ST3GAL3-CDG.

Congenital disorders of glycosylation (CDG) that may be treated with compositions of the present disclosure include, for example, Ta (PMM2-CDG), Tb (MPI-CDG), Ic (ALG6-CDG), Id (ALG3-CDG), Ie (DPM1-CDG), If (MPDU1-CDG), Ig (ALG12-CDG), Ih (ALG8-CDG), Ii (ALG2-CDG), Ij (DPAGT1-CDG), Ik (ALG1-CDG), 1L (ALG9-CDG), Im (DOLK-CDG), In (RFT1-CDG), To (DPM3-CDG), Ip (ALG11-CDG), Iq (SRD5A3-CDG), Ir (DDOST-CDG), DPM2-CDG, TUSC3-CDG, MAGT1-CDG, DHDDS-CDG, I/IIx, IIa (MGAT2-CDG), IIb (GCS1-CDG), IIe (SLC335C1-CDG), IId (B4GALT1-CDG), IIe (COG7-CDG), IIf (SLC35A1-CDG), IIg (COG1-CDG), IIh (COG8-CDG), IIi (COG5-CDG), IIj (COG4-CDG), IIL (COG6-CDG), ATP6V0A2-CDG, MAN1B1-CDG, and ST3GAL3-CDG.

In some embodiments, “treatment” or “treating” includes an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

In some embodiments, “prevention” or “preventing” includes any treatment of a disease or condition that causes the clinical symptoms of the disease or condition not to develop. Compounds may, in some embodiments, be administered to a subject (including a human) who is at risk or has a family history of the disease or condition.

In some variations, an “effective amount” is at least an amount effective, at dosages and for periods of time necessary, to achieve the desired therapeutic or prophylactic result. An effective amount can be provided in one or more administrations.

In some variations, a “therapeutically effective amount” is at least the minimum concentration required to effect a measurable improvement of a particular disease, disorder, or condition, such as a congenital disorder of glycosylation. A therapeutically effective amount herein may vary according to factors such as the disease state, age, sex, and weight of the subject, and the ability of the compositions of the present disclosure to elicit a desired response in the subject. A therapeutically effective amount is also one in which any toxic or detrimental effects of the compositions of the present disclosure are outweighed by the therapeutically beneficial effects.

In one aspect, provided herein is a method for delivering compositions of the present disclosure to a subject in need thereof. In some embodiments, the method comprises administering to the subject any of the compositions described herein.

In another aspect, provided herein is a method for delivering compositions of the present disclosure to a cell interior of a subject in need thereof. In some embodiments, the method comprises administering to the subject any of the compositions described herein. In some embodiments, at least a portion of the administered composition traverses the cell plasma membrane to deliver the carbohydrate to the cell interior.

In another aspect, provided herein is a method for treating a congenital disorder of glycosylation (CDG) in a subject in need thereof, by administering the compositions (including pharmaceutical compositions) provided herein to the subject. In some embodiments, the method comprises administering to the subject any of the compositions described herein. In some embodiments, the congenital disorder of glycosylation (CDG) is a CDG-Ia disorder. In some embodiments, the administration of the composition induces a 0.05-fold to at least a 3-fold increase in cellular production of higher-order lipid-linked oligosaccharides in the human, as compared to cellular production of higher-order lipid-linked oligosaccharides in the human in the absence of administering the composition to the subject.

### Articles of Manufacture and Kits

The present disclosure also provides articles of manufacture and/or kits containing a composition of the present disclosure. Articles of manufacture and/or kits of the present disclosure may include one or more containers comprising a composition of the present disclosure. Suitable containers may include, for example, bottles, vials, syringes, and IV solution bags. The containers may be formed from a variety of materials such as glass or plastic. In some embodiments, the articles of manufacture and/or kits further include instructions for use in accordance with any of the methods of the present disclosure. In some embodiments, these instructions comprise a description of administration of a composition of the present disclosure to deliver the carbohydrate to a subject in need thereof, to deliver the carbohydrate to a cell interior of a subject in need thereof, or to treat a congenital disorder of glycosylation (CDG) to a subject in need thereof, according to any of the methods of the present disclosure. In some embodiments, the instructions comprise a description of how to detect a congenital disorder of glycosylation (CDG), for example in a subject, in a tissue sample, or in a cell. The article of manufacture and/or kit may further comprise a description of selecting a subject suitable for treatment based on identifying whether that subject has the disease and the stage of the disease.

The instructions generally include information as to dosage, dosing schedule, and route of administration for the intended treatment. The containers may be unit doses, bulk packages (e.g., multi-dose packages) or sub-unit doses. Instructions supplied in the articles of manufacture and/or kits of the present disclosure are typically written instructions on a label or package insert (e.g., a paper sheet included in the article of manufacture and/or kit), but machine-readable instructions (e.g., instructions carried on a magnetic or optical storage disk) are also acceptable.

The label or package insert indicates that the composition is used for delivering a carbohydrate and/or treating, e.g., a congenital disorder of glycosylation (CDG). Instructions may be provided for practicing any of the methods described herein.

The articles of manufacture and/or kits of the present disclosure may be in suitable packaging. Suitable packaging includes, for example, vials, bottles, jars, and flexible packaging (e.g., sealed Mylar or plastic bags). Also contemplated are packages for use in combination with a specific device, such as an inhaler, nasal administration device (e.g., an atomizer) or an infusion device such as a minipump. An article of manufacture and/or kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). In some variations, the container may further comprise a second pharmaceutically active agent.

Articles of manufacture and/or kits may optionally provide additional components such as buffers and interpretive information. Normally, the article of manufacture and/or kit comprises a container and a label or package insert(s) on or associated with the container.

### EXAMPLES

The presently disclosed subject matter will be better understood by reference to the following Examples, which are provided as exemplary of the invention, and not by way of limitation.

### Example 1: Synthesis of Mannose-1-Phosphate

This example demonstrates the synthesis of Compound 130 on a 80+ kg scale. The steps of the synthesis in this example were performed in accordance with the scheme provided in FIG. 1. Further, a continuous flow production system is employed in steps 3 and 4, and these steps were performed in accordance with both FIG. 1 and FIG. 2; the procedures for steps 3 and 4 reference both figures.

### Step 1: Synthesis of (2R,3S,4S,5R,6R)-6-((benzoyloxy)methyl)tetrahydro-2H-pyran-2,3,4,5-tetrayl tetrabenzoate (Compound 106)

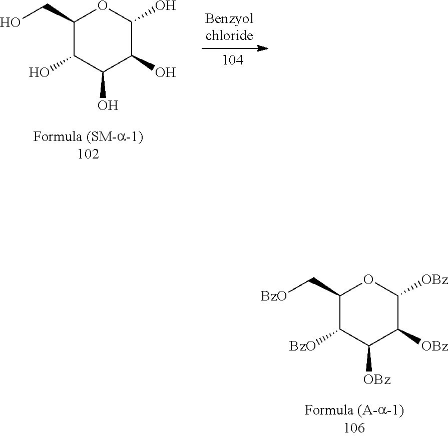

To a solution of Compound 102 (1.0 equiv) in pyridine (6V) at a temperature between 10° C. and 20° C. was added Compound 104 (benzoyl chloride, 5.5 equiv). This solution was stirred at a temperature between 20° C. and 25° C. for 3 h. The reaction mixture was then diluted with toluene (10 v) and water (10 v). The organic component was separated and washed with a 7% solution of NaHCO₃ (2×4V) and H₂O (3V), and concentrated to 5V-6V. The solvent was then exchanged with toluene (4×5V) and the mixture carried forward for use in the next step without further purification.

### Step 2: Synthesis of (2R,3R,4S,5S,6S)-2-((benzoyloxy)methyl)-6-hydroxytetrahydro-2H-pyran-3,4,5-triyl tribenzoate (Compound 110)

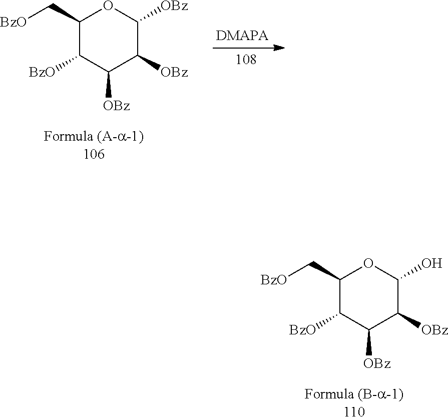

A toluene solution of Compound 106 (1.0 equiv) was diluted with THE (6V). To the reaction mixture was added DMAPA (2.0 equiv) under N₂ protection. The reaction mixture was stirred for 12-14 h at 60-65° C. and diluted with EtOAc (5V). After that, the resulting mixture was quenched with a 5% H₂SO₄ aqueous solution and separated. The organic layer was decolored via CUNO filtration, concentrated, and crystallized in methyl cyclohexane to yield Compound 110 as a white solid.

### Step 3: Synthesis of (2R,3R,4S,5S,6R)-2-((benzoyloxy)methyl)-6-((bis(benzyloxy)phosphaneyl)oxy)tetrahydro-2H-pyran-3,4,5-triyl tribenzoate (Compound 114)

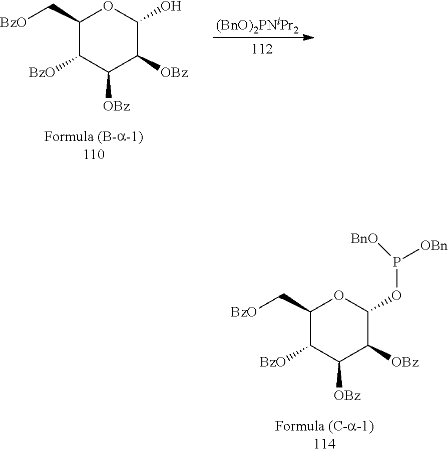

Two solutions were prepared. The first solution contained Compound 110 (1.0 equiv) and DCI (1.77 equiv) in acetonitrile (10V) at −20 to −14° C., stored in Tank 202. The second solution contained Compound 112 ((BnO)₂PNiPr₂, 1.3 equiv) in acetonitrile (10V) at RT, stored in Tank 204. A quenching solution was prepared separately, containing Na₂SO₃ (4.8 equiv) in water (10V), stored in Tank 230. The first two solutions were loaded into pre-cooling tubings 206 and 208, respectively, and combined Flow Reactor 214, for a total residence time of 1 min, and at a temperature between 0° C. and 5° C. A reaction stream comprising compound 114 is the product of this step, and is carried forward in the second flow chemistry step without isolation or purification.

### Step 4: Synthesis of (2R,3R,4S,5S,6R)-2-((benzoyloxy)methyl)-6-((bis(benzyloxy)phosphoryl)oxy)tetrahydro-2H-pyran-3,4,5-triyl tribenzoate (Compound 118)

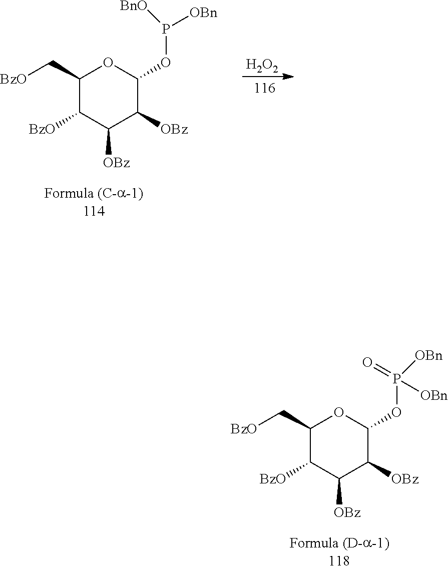

The reaction stream comprising compound 114 entered into Flow Reactor 224 and was continuously combined with oxidant 218 (H₂O₂, 3 equiv), for a residence time of 15 min, and at a temperature between 20° C. and 25° C. The resulting reaction stream was collected in Tank 228, where it was then combined with the solution of Na₂SO₃ at a temperature between 0° C. and 10° C., for a residence time between 3 and 10 min at 0-5° C. The resulting reaction mixture was diluted with toluene and entered into a centrifugal extractor immediate separation. The reaction mixture was concentrated, and then dissolved in EtOAc (3V to 4V). The resultant reaction mixture was carried forward for use in the next step without further purification.

### Step 5: Synthesis of (2R,3R,4S,5S,6R)-2-((benzoyloxy)methyl)-6-(phosphonooxy)tetrahydro-2H-pyran-3,4,5-triyl tribenzoate (Compound 126)

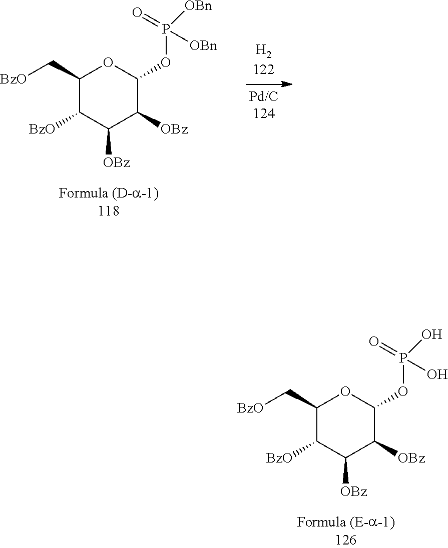

To an EtOAc solution of Compound 118 (1.0 equiv) was added MeOH (5V to 7V) in an autoclave. To the reaction mixture was added wet Pd/C (15% w/w, 0.15-0.17 equiv). The autoclave was vacuum-purged with N₂ three times, and then vacuum-purged with H₂ for three times in sequence. After stirring for 18 hours at a temperature between 20° C. and 30° C. under 45-50 psi H₂ atmosphere, the reaction mixture was filtered and the solvent switched to MeOH. Compound 126 was obtained as a yellow MeOH solution (97.7% yield), which was carried forward for use in the next step without additional purification.

### Step 6: Synthesis of potassium (2R,3S,4S,5S,6R)-3,4,5-trihydroxy-6-(hydroxymethyl)tetrahydro-2H-pyran-2-yl phosphate (Mannose-1-Phosphate potassium salt, Compound 130)

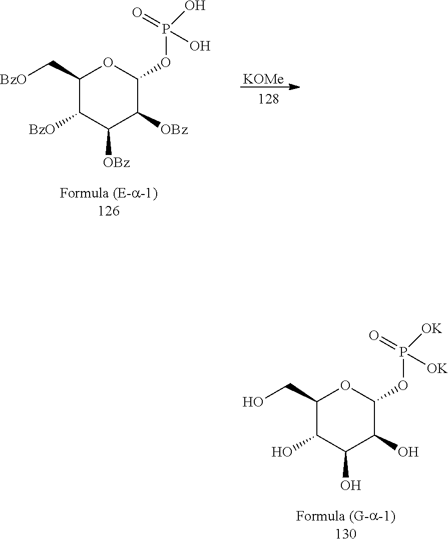

A solution of compound 126 (1.0 equiv) in MeOH (10V) was dried via solvent exchange with dry MeOH. To this solution was added a solution of KOMe (4.0 equiv) in MeOH, and the resultant reaction mixture was stirred at a temperature between 20° C. and 25° C. under N₂ atmosphere for 20-22 hours. The solid product was then isolated via filtration, taken up again in MeOH (3V) to produce a slurry, and then filtered again to yield a crude white solid. This solid was recrystallized in H₂O:MeOH to obtain a hydrate of Compound 130 (60-70% yield), as a white solid. Compound 130 can then be subjected to further crystallization procedures to generate Form C, as described in Example 6 below.

### Example 2: Instability of Compound 110

In this invention, the use of flow chemistry techniques was essential in producing a final product that was substantially pure in the desired alpha isomer of M1P. This is because under the conditions required for the transformation of Compound 110 to Compound 118, a significant amount of isomerization of the reaction intermediates, from the alpha to the beta form, occurs over time. The flow chemistry techniques described herein lessen the amount of isomerization in this transformation.

Table 1 demonstrates the instability of Compound 110 over time in solution. When the reaction solution comprising 110 and DCI is stored, and not used immediately following its preparation, the amount of beta isomer of product 118 increases.

TABLE 1
Instability of Compound 110 in Reaction Solution

| Solution 1 | | | HPLC results (% | | |
|---|---|---|---|---|---|
| Compound | Solution 2 | | following transformation | | |
| 110 and DCI | (BnO)₂PNiPr₂ | Reaction | of 110 to 118) | | |
| in MeCN | in MeCN | Conditions | 110 | 118 | β isomer |
| Fresh used | Fresh used | −15° C./ | 0.9 | 94.5 | 4.2 |
| | | 10 min | | | |
| Stored for | Stored for | −15° C./ | 0.2 | 90.4 | 8.5 |
| 20 h | 20 h | 10 min | | | |
| Stored for | Fresh used | −15° C./ | 0.2 | 90.1 | 8.5 |
| 20 h | | 10 min | | | |
| Stored for | Stored for | −15° C./ | 0.6 | 90.0 | 9.1 |
| 44 h | 44 h | 10 min | | | |
| Stored for | Fresh used | −15° C./ | 0 | 90.4 | 9.3 |
| 44 h | | 10 min | | | |

### Example 3: Batch Synthesis Vs. Flow Chemistry Synthesis of Compound 118

This example compares the production of Intermediate D using a batch synthesis as compared to flow chemistry synthesis in accordance with the procedures set forth in Example 1 above.

For the batch synthesis, Compound 110 (1.0 equiv) and DCI (1.77 equiv) were dissolved in acetonitrile (10V). To this solution was added (BnO)₂PNⁱPr₂ (1.5 equiv), and the resultant mixture was stirred at −20 to −15° C., to generate Compound 114. Upon completion of reaction, BuOOH (1.6-1.8 equiv) was added to the reaction mixture, which stirred at −10 to −5° C. to generate Compound 118. The reaction mixture was washed with 10% citric acid followed by 7% NaHCO₃, and then purified via silica gel chromatography to remove the undesired beta isomer of 118. On a 100 g scale, this procedure successfully yielded 118 with 95% purity, and with only 2-3% of the beta isomer of 118 present as an impurity. However, at larger (25 kg) scale, the amount of beta isomer present increased up to 15%. This highlights the importance of the flow chemistry techniques described herein, which allow for production of substantially pure alpha isomers of 118 at large scale, and thus, large scale amounts of M1P in substantially pure alpha isomeric form.

### Example 4: Preparation and Characterization of Polymorphic Form A of M1P (Potassium Hydrate Salt)

This example demonstrates the synthesis and characterization of polymorphic Form A of M1P, which is a dipotassium dihydrate salt.

M1P Form A was prepared using the following procedure. About 500 mg of M1P was added into a vial. 3 mL of MeOH was added into the vial under stirring at 5° C. After stirring at 5° C. for about 5 days, the resulting suspension was filtered. The obtained solids were air-dried under ambient conditions for about 22 hours. About 420 mg of the M1P Form A was obtained as a white solid in 84% yield. See Table 2 for characterization data of Form A.

TABLE 2
Characterization of Polymorphic Form A of M1P

| Parameter | Method | Result |
|---|---|---|
| Purity | HPLC | 97.1% |
| X-ray diffraction | XRPD, 3-40° (2 theta) | High crystallinity |
| Melting onset | DSC, 10° C./min | Dehydration onset: 88.8° C. |
| and enthalpy | | No melting peak appears after dehydration |
| | | Dehydration enthalpy: ~314 J/g |
| Thermogravimetry | TGA, 10° C./min | 9.9% @ 150° C. |
| Residual solvents | ¹H-NMR(D₂O) | No residual solvent |
| Water content | Karl Fisher | 10.3% water by weight (equal to 2.1 equiv.) |
| Counter ion(s) | Ion chromatography | Free form:K = 1:2.02 |
| Morphology and | SEM | Plate-like crystal, particle size: 1-20 μm |
| particle size | | |

### Example 5: Preparation and Characterization of Polymorphic Form B of M1P (Potassium Hydrate Salt)

This example demonstrates the synthesis and characterization of polymorphic Form B of M1P, which is a dipotassium trihydrate salt.

M1P Form B was prepared using the following procedure. About 500 mg of M1P was dissolved in 1.5 mL of water at ambient temperature. This solution was filtered through a syringe with a 0.45 μm membrane filter. 1.5 mL of the filtered solution was quickly added into 7.5 mL of MeOH in a 20 mL glass vial. The clear solution immediately became a suspension, and this suspension was stirred at 25° C. After stirring at 25° C. for about 6 days, the suspension was filtered through a 0.45 μm nylon membrane filter at 4,000 rpm. Obtained solids were air-dried at ambient condition for about 22 hours. About 360 mg of the M1P Form B was obtained as a white solid in 72% yield. See Table 3 for characterization data of Form B.

TABLE 3
Characterization of Polymorphic Form B of M1P

| Parameter | Method | Result |
|---|---|---|
| Purity | HPLC | 98.1% |
| X-ray diffraction | XRPD, 3-40° (2 theta) | High crystallinity |
| Melting onset | DSC, 10° C./min | Dehydration onset: 35.8° C., 62.7° C., 96.6° C. |
| and enthalpy | | No melting peak appears after dehydration |
| | | Dehydration enthalpy: ~9 J/g, ~14 J/g, ~152 J/g |
| Thermogravimetry | TGA, 10° C./min | 4.2% @ 60° C., 2.9% @ 60-85° C., 6.5% @ 85-150° C. |
| Residual solvents | ¹H-NMR (D₂O) | No residual solvent |
| Water content | Karl Fisher | 13.0% water by weight (equal to 2.8 equiv.) |
| Counter ion(s) | Ion chromatography | IC:Stoichiometric ratio: free form:K⁺ = 1:2.05 |
| Morphology and | SEM | Plate-like crystal, particle size: 1-50 μm |
| particle size | | |

### Example 6A: Preparation and Characterization of Polymorphic Form C of M1P (Potassium Hydrate Salt)

This example demonstrates the synthesis and characterization of polymorphic Form C of M1P, which is a dipotassium trihydrate salt.

M1P Form C was prepared using the procedure below. About 500 mg of M1P was added into an 8 mL glass vial. 3 mL of water:MeOH (1:10, v/v) was added into the vial under stirring at 25° C., which resulted in a suspension. About 5 mg of Form C seeds were added into the suspension. After stirring at 25° C. for about 6 days, the suspension was filtered through a 0.45 μm nylon membrane filter at 4,000 rpm. The obtained solids were air-dried under ambient conditions for about 22 hours. About 389 mg of the M1P Form C was obtained as a white solid in 77% yield.

M1P Form C was also prepared using the following process. M1P potassium salt (0.97-1.03 equiv) was dissolved in water (2.3-2.4V) in a reactor. An additional amount of water (0.2-1.6V) was added to the reactor via a pray header, and the temperature of the reactor was adjusted to between 2° and 30° C. The solution was stirred for 1-2 hours until clear. The temperature was adjusted to 0 to 10° C., and then MeOH (2.5-3.0V) was slowly added. M1P Form C seed crystals (0.1%-2.0%×) were added, and the mixture stirred at 0 to 10° C. for 14-20 h. Then, H₂O:MeOH (Volume ratio 1/3, 2.5-3.0× (target 2.7×)) was slowly added at 0 to 10° C. over 4 hours, and stirring continued for 6 to 8 hours. Stirring continued and additional MeOH was added until a sample taken for XRPD analysis demonstrated accordance with an internal standard of Form C. Finally, the crystals were isolated via active filter drying (AFD).

TABLE 4a
Characterization of Polymorphic Form C of M1P

| Parameter | Method | Result |
|---|---|---|
| Purity | HPLC | 98.6% |
| X-ray diffraction | XRPD, 3-40° (2 theta) | High crystallinity |
| Melting onset | DSC, 10° C./min | Dehydration onset: 85.5° C., 119.7° C. |
| and enthalpy | | No melting peak appears after dehydration |
| | | Dehydration enthalpy: ~5 J/g, ~172 J/g |
| Thermogravimetry | TGA, 10° C./min | 5.0% @ 101° C., 7.6% @ 101-150° C. |
| Residual solvents | ¹H-NMR (D₂O) | No residual solvent |
| Water content | Karl Fisher | 14.4% water by weight (equal to 3.1 equiv.) |
| Counter ion(s) | Ion chromatography | Free form:K = 1:2.05 |
| Morphology and | SEM | Irregular crystal, particle size: 1-20 μm |
| particle size | | |

### Example 6B: Polymorph Screening

This example describes a polymorph screen that was conducted using several different solvent systems and various conditions. Overall, it was surprisingly observed that only certain solvent systems and certain conditions led to production of M1P Form C (as described in Example 6A above).

Equilibration with solvents at 5° C. for 2 weeks: About 50-150 mg of M1P (mixture of Form A and Form B) was equilibrated in 0.04-0.40 mL of the following solvents at 5° C. for 2 weeks with stirring: water, methanol (MeOH), ethanol (EtOH), isopropanol, acetone, methyl ethyl ketone, ethyl acetate, acetonitrile, isopropyl acetate (IPAc), tetrahydrofuran (THF), dichloromethane (DCM), 2-methylTHF, and water/methanol at various ratios (10:1, 5:1, 3:2, 1:1, 2:3, 1:2, 1:5 and 1:10, v:v). Based on XRPD, Form C was only observed using water/methanol at 5:1, 3:2, 1:1, 2:3, 1:2, and 1:10, v:v. No XRPDs were obtained for the water and water/MeOH (10:1, v:v) samples. XPRDs for the other remaining samples showed Form A, Form B, or a mixture of Form A and Form B.

Equilibration with solvents at 25° C. for 1 week: About 50-150 mg of M1P (mixture of Form A and Form B) was equilibrated in 0.04-0.50 mL of the following solvents at 25° C. for 1 week with stirring: water, methanol (MeOH), ethanol (EtOH), isopropanol, acetone, methyl ethyl ketone, ethyl acetate, acetonitrile, isopropyl acetate (IPAc), tetrahydrofuran (THF), dichloromethane, 2-methylTHF, and water/methanol at various ratios (10:1, 5:1, 3:2, 1:1, 2:3, 1:2, 1:5 and 1:10, v:v). Based on XRPD, Form C was only observed using water/methanol at 5:1, 3:2, 1:1, 2:3, 1:2, and 1:10, v:v. No XRPDs were obtained for the water and water/MeOH (10:1, v:v) samples. XPRDs for the other remaining samples showed Form B, or a mixture of Form A and Form B.

Equilibration under a temperature cycle: About 50-150 mg of M1P (mixture of Form A and Form B) was equilibrated in 0.04-0.40 mL of the following solvents under a temperature cycle between 5° C. to 50° C. at a heating/cooling rate of 0.1° C./min for 10 cycles, and equilibration was achieved under stirring: water, methanol (MeOH), ethanol (EtOH, isopropanol, acetone, methyl ethyl ketone, ethyl acetate, acetonitrile, isopropyl acetate (IPAc), tetrahydrofuran (THF), anisole, 2-methylTHF, and water/methanol at various ratios (10:1, 5:1, 3:2, 1:1, 2:3, 1:2, 1:5 and 1:10, v:v). Based on XRPD, Form C was only observed using water/methanol at 3:2, 1:1, and 1:2, v:v. No XRPDs were obtained for the water, and water/MeOH (10:1 and 5:1, v:v) samples because the M1P was too soluble. XPRDs for the other remaining samples showed Form A, or a mixture of Form A and Form B.

Crystallization from hot saturated solutions by slow cooling: About 50 mg of M1P (mixture of Form A and Form B) was dissolved in the minimal amount of the following solvents at 50° C.: Water/MeOH (1:1, v:v), Water/EtOH (1:1, v:v), Water/IPA (8:2, v:v), Water/ACN (8:2, v:v), Water/acetone (8:2, v:v), and Water/THF (8:2, v:v). Obtained solutions were filtered, and the clear solutions were cooled to 5° C. at 0.1° C./min. Samples without precipitates at 5° C. were further cooled to −20° C. XRPDs were not carried out for the samples above because the M1P was too soluble.

Crystallization from hot saturated solutions by fast cooling: About 50 mg of M1P (mixture of Form A and Form B) was dissolved in the minimal amount of the following solvents at 50° C.: Water/MeOH (1:1, v:v), Water/EtOH (1:1, v:v), Water/IPA (8:2, v:v), Water/ACN (8:2, v:v), Water/acetone (8:2, v:v), and Water/THF (8:2, v:v). Obtained solutions were filtered, and the clear solutions were put into a 0° C. ice bath and agitated. Samples without precipitates at 5° C. were further cooled to −20° C. XRPD was only carried out for Water/MeOH (1:1, v:v), which showed Form C. No XRPDs were taken for the rest of the samples above because the M1P was too soluble.

Crystallization by addition of anti-solvent: About 1 g of M1P (mixture of Form A and Form B) was dissolved in 2 mL of water at ambient temperature (about 20-22° C.). Obtained solutions were filtered. Then, 0.1 mL of clear solution was pipetted into a vial to which 3-5 folds of the anti-solvents tested were slowly added into the clear solutions. Samples without precipitates were further cooled to −20° C. The anti-solvents tested include: methanol (MeOH) (0.3 mL), ethanol (EtOH) (0.3 mL), isopropanol (IPA) (0.3 mL), acetone (0.5 mL), methyl ethyl ketone (MEK) (0.5 mL), acetonitrile (ACN) (0.5 mL), tetrahydrofuran (THF) (0.5 mL), 2-methylTHF (0.5 mL), and 1,4-dioxane (0.5 mL). Precipitates were collected by centrifugation filtration. Solid parts (wet cakes) were investigated by XRPD. XRPD was obtained only for water/methanol system, which showed Form B. No XRPDs were taken for the rest of the samples above because the M1P was too soluble.

Crystallization by reverse addition of anti-solvent: About 1 g of M1P (mixture of Form A and Form B) was dissolved in 2 mL of water at ambient temperature (about 20-22° C.). Obtained solutions were filtered. Then, 0.1 mL of clear solution was added into 5 folds of the anti-solvents tested in a vial. Samples without precipitates were further cooled to −20° C. The anti-solvents tested include: methanol (MeOH), ethanol (EtOH), isopropanol (IPA), acetone, methyl ethyl ketone (MEK), acetonitrile (ACN), tetrahydrofuran (THF), 2-methylTHF, and 1,4-dioxane. Precipitates were collected by centrifugation filtration. Solid parts (wet cakes) were investigated by XRPD. For samples with different XRPD patterns, additional analysis including DSC, TGA, IC, KF, ¹H-NMR and PLM was performed. Based on the XRPDs, Form B was observed to be obtained in water/methanol, water/ethanol, water/isopropanol, water/acetone and water/1,4-dioxane. No XRPDs were taken for the rest of the samples above because the M1P was too soluble.

Crystallization by vapor diffusion: About 500 mg of M1P (mixture of Form A and Form B) was dissolved in 0.5 mL water at ambient temperature (about 20-22° C.). Obtained solutions were filtered. Then, clear solutions were transferred into small vials without lids. The small lidless vials were placed in larger vials. Then, the anti-solvents tested were added to the larger vials. The anti-solvents tested include: methanol (MeOH), ethanol (EtOH), acetone, methyl ethyl ketone (MEK), heptane, methyl tert-butyl ether (MTBE), tetrahydrofuran (THF), 2-methylTHF, and acetonitrile (ACN). Then the larger vials were capped tightly and placed at ambient condition. Precipitates were collected by centrifugation filtration. Solid parts (wet cakes) were investigated by XRPD. XRPDs of the water/methanol sample and the water/acetone sample showed a mixture of Form A and Form B. XPRD of water/ethanol showed Form A. No XRPDs were taken for the rest of the samples above because the M1P was too soluble.

### Example 7: Water Activity Experiments

This example demonstrates that M1P Form C is stable over a wide water activity range. Generally, Form C is the more stable form as compared to Form A and Form B. Further, Form A and Form B convert to Form C under certain specific conditions. Water activity experiments were conducted at 25° C. in several different water/EtOH systems to determine relative stability of M1P Forms A, B and C under these conditions.

About 5 mg each of M1P Forms A, B, and C were combined and added to 0.2 mL saturated solutions of a water/EtOH system. The resulting suspensions were stirred at 5° C., 25° C., or 50° C. for 4 days or 11 days. After 4 or 11 days, the solids (wet cakes) were isolated by centrifugation filtration and analyzed by XRPD to determine which Form(s) remained. The results are summarized in Table 5.

TABLE 5
Water Activity Experiments to Determine Relative Stability of Forms A,
B, and C in Solvent Systems with Varying Water Activity (a.w) Values

| | XRPD Form Observed | | | | | |
|---|---|---|---|---|---|---|
| | 4 days | 11 days | 4 days | 11 days | 4 days | 11 days |
| Solvent System | at 5° C. | at 5° C. | at 25° C. | at 25° C. | at 50° C. | at 50° C. |
| Water/EtOH (30:70, | Form C | // | Form C | // | Form C | // |
| v:v), a.w. = 0.8 | | | | | | |
| Water/EtOH (20:80, | Form C | // | Form C | // | Form C | // |
| v:v), a.w. = 0.7 | | | | | | |
| Water/EtOH (14:86, | Form C + | Form C | Form C | // | Form C | // |
| v:v), a.w. = 0.6 | a trace | | | | | |
| | amount of | | | | | |
| | Form A | | | | | |
| Water/EtOH (10:90, | Form C | // | Form C | // | Form C | // |
| v:v), a.w. = 0.5 | | | | | | |
| Water/EtOH (08:92, | Form C | // | Form C | // | Form A + | Form C + |
| v:v), a.w. = 0.4 at | | | | | Form C | a trace |
| 5° C.; | | | | | | amount of |
| Water/EtOH (07:93, | | | | | | Form A |
| v:v), a.w. = 0.4 at | | | | | | |
| 25° C. and 50° C. | | | | | | |
| Water/EtOH (05:95, | Form C | // | Form A + | Form C + | Form A + | Form C + |
| v:v), a.w. = 0.3 | | | Form C | a trace | Form C | a trace |
| | | | | amount of | | amount of |
| | | | | Form A | | Form A |
| Water/EtOH (03:97, | Form C | // | Form A + | Form A + | Form A + | Form A |
| v:v), a.w. = 0.2 | | | Form C | Form C | Form C | |
| Water/EtOH (01:99, | Form A + | Form A + | Form A + | Form A + | Form A + | Form A + |
| v:v), a.w. = 0.1 | Form C | Form C | Form C | Form C | Form C | Form C |
| EtOH, a.w. = 0 | Form A + | Form A + | Form A + | Form A + | Form A + | Form A |
| | Form C | Form C | Form C | Form C | Form C | |

Note:
water activity is calculated by UNIFAC method.
“//”: Not carried out.

### Example 8: Water Sorption and Desorption Experiments

Water sorption behavior of M1P Form C was investigated using dynamic vapor sorption (DVS) at 25° C. The relative humidity (RH) was increased from 0-80%, with a minimum equilibration time of 60 min and a maximum equilibration time of 360 min. As shown in Table 6, Form C absorbed only 1.9% (w/w) H₂O at 80% RH. XRPD analysis was performed following the DVS experiment, and no form change was observed, demonstrating the low hygroscopicity and favorable stability of Form C.

TABLE 6
DVS Experiments with Form C at 25° C.

| Relative humidity | % Water absorption |
|---|---|
| 0% | 0.01 |
| 10% | 0.51 |
| 20% | 0.59 |
| 30% | 0.63 |
| 40% | 0.65 |
| 50% | 0.69 |
| 60% | 0.73 |
| 70% | 0.82 |
| 80% | 1.93 |

### Example 9: Stability of Form C

This example illustrates the stability of crystalline Form C in several stress tests. Form C was subjected to compression, dry grinding simulation, and wet granulation simulation. The results are summarized in Tables 7-9.

### Compression Study

About 10 mg of M1P Form C was compressed for 5 minutes under 5 MPa or 10 MPa with a hydraulic press. Potential form change and degree of crystallinity were evaluated by XRPD. Based on the XPRD results from the compression study, no form change was observed from compression at 5 MPa and 10 MPa, and crystallinity decreased by showing broad peaks.

### Dry Grinding Study

About 10 mg of M1P Form C was ground manually with a mortar and a pestle for 3 min. Potential form change and degree of crystallinity were evaluated by XRPD. The XPRD results from the dry grinding study showed no form change after 3 min of grinding time, and crystallinity decreased by showing broad peaks.

### Wet Granulation Simulation

Ethanol was added dropwise to about 10 mg of M1P Form C until the sample was wetted sufficiently. The wet sample was ground gently in a mortar and a pestle. Post granulation, the sample was dried under ambient conditions for 10 min. Potential form change and degree of crystallinity were evaluated by XRPD. The XRPD results from the wet granulation study showed no form change in ethanol as the granulation solvent.

### Example 10: Solubility of M1P Form C

This example demonstrates solubility of M1P Form C. 242.1 mg of M1P Form C, (equivalent to 160 mg of the free base of M1P), was weighed into a 2 mL glass vial. 1 mL of solubility medium (pH 7.0 Tris buffer, 50 mM) was added. The obtained solution was stirred at 37° C. and 400 rpm for 2 h and 24 h, and was then centrifuged at 37° C. and 14,000 rpm for 5 min. At each of the 2 h and 24 h timepoints, the supernatant was analyzed by pH meter to determine solubility. M1P Form C demonstrated favorable solubility of over 160 mg/mL.

### Example 11: Polymorph Characterization of M1P Form C Through 12-Month Stability

M1P Form C was produced in accordance with the examples above. XRPD was used on samples taken at 1, 3, 6, 9 and 12 months, and tested under four conditions:
−20° C.±5° C.,
25° C.±2° C. at 60% relative humidity (RH)±5% RH,
40° C.±2° C. at 75% RH±5% RH and
5° C.±3° C.

In each instance, the XRPD of the samples corresponded to the reference standard spectra for M1P Form C, indicating that the samples remained as Form C at all timepoints tested. There were no observed changes to the polymorphic form at any temperature condition under study.

Further, there were no substantial changes observed with respect to the purity of Form C as determined based on chromatography.

1. A continuous flow production method, comprising:
a) providing a compound of Formula B-α-1 and a nucleophilic catalyst at a temperature between −30° C. and −10° C. to yield a precooled solution, wherein the compound of Formula B-α-1 is: 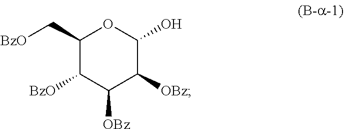
b) continuously combining the precooled solution and a phosphorylating agent in a first reactor at a temperature between −30° C. and 5° C. and a residence time between 30 seconds and 5 minutes, to produce an intermediate composition comprising a compound of Formula (C-α-1): 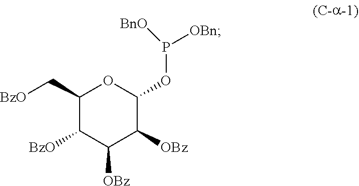
c) transferring the intermediate composition from the first reactor to a second reactor; and
d) continuously combining the intermediate composition with an oxidant in the second reactor at a temperature between 0° C. and 30° C. and for a residence time between 10 minutes and 30 minutes to produce a reaction mixture comprising a compound of Formula (D-α-1):

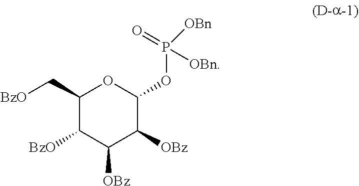

2-30. (canceled)

31. A composition comprising an alpha isomer of mannose-1-phosphate, or a salt thereof, or a hydrate of any of the foregoing, wherein the purity of the composition is at least 96%.

32-36. (canceled)

37. A method of producing a crystalline Form C of mannose-1-phosphate, comprising:
a) combining a starting composition comprising (i) substantially pure alpha isomer of mannose-1-phosphate, or a salt thereof, or a hydrate of any of the foregoing, or (ii) crystalline Form A of a substantially pure alpha isomer of mannose-1-phosphate, or a salt thereof, or a hydrate of any of the foregoing, or (iii) crystalline Form B of a substantially pure alpha isomer of mannose-1-phosphate, or a salt thereof, or a hydrate of any of the foregoing, or any combination of (i)-(iii), with a solvent comprising water and alcohol to produce a suspension; and
b) stirring the suspension to produce a product composition comprising crystalline Form C of mannose-1-phosphate.

38-42. (canceled)

43. A crystalline Form C of mannose-1-phosphate that is a substantially pure alpha isomer of a potassium trihydrate salt of mannose-1-phosphate.

44. The crystalline Form C of claim 43, exhibiting an X-ray powder diffraction (XRPD) pattern comprising peaks at 11.6, 14.6, 17.8, 23.2, 27.7, and 30.7±0.2 degrees 2-theta.

45. The crystalline Form C of claim 43, exhibiting an XRPD substantially similar to FIG. 1.

46. The crystalline Form C of claim 43, exhibiting a TGA thermogram with a weight loss of about 5% to 8% in the temperature range of 101-150° C.

47. The crystalline Form C of claim 43, exhibiting a TGA thermogram substantially similar to FIG. 6.

48. The crystalline Form C of claim 43, having an average particle size between 1 μm and 20 μm.

49. The crystalline Form C of claim 43, wherein:
(i) when compressed for 5 minutes under 5 MPa and/or under 10 MPa, is stable; or
(ii) when ground, whether under dry conditions or wet conditions, is stable; or
(iii) when stored for one week in an open container at 40° C. and 75% relative humidity, or for one week in an open container at about 40° C. and about 75% relative humidity, is stable; or
(iv) when stored for:
one week in a closed container at about 60° C.;
up to about twelve months at about −20° C.;
up to about twelve months at about 25° C. and about 60% relative humidity;
up to about twelve months at about 5° C.; and/or
up to about twelve months at about 40° C. and about 75% relative humidity, is stable;
or any combination of (i)-(iv).

50. (canceled)

51. (canceled)

52. (canceled)

53. A composition, comprising: at least 98% by weight of a crystalline Form C according to claim 43.

54. The composition of claim 53, wherein less than 1% of the composition is the beta isomer of mannose-1-phosphate potassium salt having the structure:

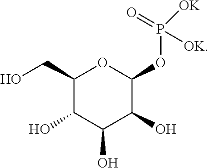

55. The composition of claim 53, wherein less than 0.5% of the composition is Form A and/or Form B of mannose-1-phosphate.

56. The composition of claim 55, wherein:
Form A of mannose-1-phosphate exhibits an XRPD pattern comprising peaks at 20.3, 21.1, 23.5, 27.1, 28.1, and 29.0±0.2 degrees 2-theta; and
Form B of mannose-1-phosphate exhibits an XRPD pattern comprising peaks at 8.3, 14.2, 19.8, 21.4, 23.9, and 27.5±0.2 degrees 2-theta.

57. A composition, comprising:
(i) a liposome; and
(ii) alpha isomer of mannose-1-phosphate, or a salt thereof, or a hydrate of any of the foregoing, encapsulated in the liposome.

58. The composition of claim 57, wherein the liposome comprises one or more phospholipids conjugated to polyethylene glycol (PEG).

59. A composition, comprising:
liposomes having a lipid membrane enclosing an intraliposomal compartment, wherein the liposomes encapsulate in the intraliposomal compartment an alpha isomer of mannose-1-phosphate, or a salt thereof, or a hydrate of any of the foregoing, and
wherein the lipid membrane comprises:
(a) at least one phospholipid having an ethanolamine head group and at least one unsaturated fatty acid tail;
(b) at least one phospholipid having a choline group and at least one unsaturated fatty acid tail; and
(c) at least one phospholipid having an ethanolamine head group and at least one saturated fatty acid tail, and
intraliposomal buffer comprising a buffer salt and optionally acid, wherein the pKa of the buffer salt is between 6 to 8.5; and
extraliposomal buffer comprising a buffer salt and a tonicity modifier, wherein the pKa of the buffer salt is between 6 to 8.5.

60. The composition of claim 59, wherein the lipid membrane comprises:
(a) at least one phospholipid having an ethanolamine head group and at least one unsaturated fatty acid tail;
(b) at least one phospholipid having a choline group and at least one unsaturated fatty acid tail; and
(c) at least one phospholipid having an ethanolamine head group and at least one saturated fatty acid tail, conjugated to polyethylene glycol (PEG).

61. (canceled)

62. The composition of any one claims 57-6, claim 57, wherein the an alpha isomer of mannose-1-phosphate, or a salt thereof, or a hydrate of any of the foregoing is α-D(+)mannose-1-phosphate dipotassium salt.

63. The composition of claim 57, wherein less than 1% of the composition is the beta isomer of mannose-1-phosphate, or a salt thereof, or a hydrate of any of the foregoing.

64. (canceled)

65. The composition of claim 64, wherein less than 0.5% of the composition is Form A and/or Form B of mannose-1-phosphate.

66. The composition of claim 57, formulated for intravenous administration, or injection.

67. (canceled)

68. The composition of claim 57, wherein:
(i) after the composition is stored at controlled room temperature, the purity of the alpha isomer of mannose-1-phosphate in the composition is at least about 98%; or
(ii) after the composition is stored at ambient temperature, the purity of the alpha isomer of mannose-1-phosphate in the composition is at least about 95%; or
a combination of (i) and (ii).

69. (canceled)

70. The compositions of claim 53, further comprising: at least one pharmaceutically acceptable carrier, excipient, and/or stabilizer.

71. A method of treating a congenital disorder of glycosylation (CDG) in a subject in need thereof, by administering the composition of claim 43.
